(19) United States
(12) Patent Application Publication
(10) Pub. No.: US 2022/0354897 A1
(43) Pub. Date: Nov. 10, 2022

(54) INTRANASALLY ADMINISTERED NEURAL STEM CELLS FOR TREATMENT OF TRAUMATIC BRAIN INJURIES

(71) Applicants: CITY OF HOPE, Duarte, CA (US); UNIVERSITY OF PITTSBURGH - OF THE COMMONWEALTH SYSTEM OF HIGHER EDUCATION, Pittsburgh, PA (US)

(72) Inventors: Margarita GUTOVA, Duarte, CA (US); Anthony KLINE, Pittsburgh, PA (US)

(73) Assignees: CITY OF HOPE, Duarte, CA (US); UNIVERSITY OF PITTSBURGH - OF THE COMMONWEALTH SYSTEM OF HIGHER EDUCATION, Pittsburgh, PA (US)

(21) Appl. No.: 17/662,627

(22) Filed: May 9, 2022

Related U.S. Application Data
(60) Provisional application No. 63/186,013, filed on May 7, 2021.

(51) Int. Cl. A61K 35/30; A61K 9/00; A61P 25/00

(52) U.S. Cl. CPC A61K 35/30; A61K 9/0043; A61P 25/00

(57) ABSTRACT

This disclosure relates to methods for treating a subject having a traumatic brain injury (TBI) by intranasal administration of neural stem cells (NSCs), while optionally providing environmental enrichment (EE) to the subject in conjunction with or in combination with the NSC administration.

### CROSS-REFERENCE TO RELATED APPLICATION

This application claims the benefit of U.S. Patent Application No. 63/186,013, filed May 7, 2021, the disclosure of which is hereby incorporated by reference in its entirety as if fully set forth herein, including all references and appendices submitted therewith.

### STATEMENT REGARDING FEDERALLY SPONSORED RESEARCH

This invention was made with government support under Grant No. 1R56NS113810-01, awarded by the National Institutes of Health. The government has certain rights in the invention.

### BACKGROUND

Traumatic brain injury (TBI) often results in long-term neurological disabilities (Selassie et al 2008; Goldstein 1990; Faul et al. 2010) and affects ten million individuals worldwide each year (Hyder et al. 2007). In the United States, the incidence is 2.8 million per year (Taylor et al. 2017b). Although motor function is negatively impacted, pre-clinical and clinical evidence indicates that cognitive impairments are more pronounced and prolonged (Kline et al. 2001; Kline et al. 2002c; Kline et al, 2007; Kline et al. 2010; Horneman & Emanuelson 2009; Bondi et al. 2014; Bogdanova et al. 2016), and adversely affect quality of life (Binder 1986; Millis et al. 2001). The economic cost of acute medical care and subsequent rehabilitation, along with productivity losses due to the inability to return to full-time work, is billions of dollars each year (Faul et al. 2010). Because TBI is a significant health care concern, numerous pharmacological interventions have been evaluated (Parton & Husain 2005; Kokiko & Hamm 2007; Wheaton et al. 2009; Garcia et al. 2011). Although various approaches have shown benefits in the laboratory, successful clinical translation has been essentially nonexistent (Doppenberg et al. 2004; Menon 2009). This dismal situation has motivated development of other alternative therapeutic strategies.

Neural stem cells (NSCs) are attractive candidates for restoring brain function after TBI as they inherently migrate to damaged sites, where they contribute neurotrophic factors to suppress inflammation, protect against further neuronal loss, promote recovery of existing damaged neurons, and possibly replace lost neurons and other cells (Gage et al. 1995; Gutova et al. 2013a; Balyasnikova et al. 2014; Aboody et al. 2013). A major challenge to successful NSC-based therapy is ensuring that sufficient numbers of cells reach damaged and non-functioning regions. To address this issue, delivery of NSCs to the central nervous system (CNS) has been explored by intravenous (IV) and intracranial (IC) injection, and by intranasal (IN) inhalation (Gutova et al. 2015; Gutova et al. 2013b; Barish et al. 2017). Although IV injected NSCs localize to damaged tissue, they show limited accumulation (less than 1% of injected NSCs) in the brain (Barish et al. 2017; Loebinger et al; 2009). IV administration of NSCs can also trigger adverse immune responses and other systemic complications. IC administration of NSCs avoids potential systemic reactions (Barish et al. 2017) but it is invasive, inefficient, costly, potentially damaging to normal brain, and places patients at greater risk. The inherent limitations of IV and IC administration support evaluation is less invasive and can be performed repeatedly (Balyasnikova et al. 2014). Thus, less invasive delivery methods and improved efficacy of the treatment is needed.

### SUMMARY

A major obstacle to successful treatment of Traumatic Brain Injury (TBI) is the blood-brain barrier, which prevents most therapeutic agents from entering the brain in amounts sufficient to treat TBI. Neural stem cells (NSCs) offer a potential solution to treating TBI by passing the blood-brain barrier and promoting cell replacement, neuroprotection and immunomodulatory effects to repair the damaged brain tissue. The studies discussed herein use a rat model of TBI to test well characterized L-MYC expressing NSC (LM-NSC008) cell migration, distribution at the TBI sites and therapeutic efficacy alone or in combination with environmental enrichment to accelerate clinical translation of NSC-based therapies to patients with TBI and other neurodegenerative diseases.

### BRIEF DESCRIPTION OF THE DRAWINGS

This application contains at least one drawing executed in color. Copies of this application with color drawing(s) will be provided by the Office upon request and payment of the necessary fees.

FIG. 1 illustrates delivery of NSCs to the brain to track migration according to some embodiments.

FIG. 2 is an image showing L-myc expressing human neural stem cells used in accordance with the embodiments described herein.

FIG. 3 illustrates the use of CLARITY to visualize NSCs.

FIGS. 4A-4H. Quantification of LM-NSC008 cell within boxes 1-7 at day 25. (4A) Section is from day 25 post CCI (LM-NSCOO8s pseudo colored green, brain outlined blue). TBI site is shown in the upper right quadrant (A* inset black and white image). (4B-4H) Number of cell clusters in boxes 1-7 (Y-axis) versus distance of LM-NSC008 cells migrated from TBI site (X-axis).

FIGS. 5A-5I Number of cell clusters quantified in each quadrant 1-9 (Y-axis) vs. distance of LM-NSC008 cells migrated from TBI site at day 46 post TBI (X-axis).

FIGS. 6A, 6B, and 6C are clean confocal images of TBI-specific distribution of intranasally delivered LM-NSC008 cells in rat brains (day 25) according to one embodiment. Details relating to an analysis of these images are discussed below in FIGS. 8A-8C.

FIGS. 7A, 7B, 7C, 7D are clean confocal images of TBI-specific distribution of intranasally delivered LM-NSC008 cells in rat brains (day 46). Details relating to an analysis of these images are discussed below in FIGS. 9A-9D.

FIGS. 8A-8C. TBI-specific distribution of intranasally delivered LM-NSC008 cells in rats (day 25). (8A) Confocal image (stitched at maximum intensity) of optically cleared coronal brain slice (1-mm thick). Rats received either a TBI or sham injury and on days 7, 9, 11, 13, 15, and 17 post-surgery were IN administered LM-NSC008 cells (1×10⁶ cells in 24 μL). On day 25, the rats were euthanized, and 1-mm brain slices were cleared using CLARITY (PACT) and imaged using confocal microscopy (8A, 8B, 8C). Three coronal sections (S1, S2, S3) are shown overlapping the TBI site. LM-NSC008 cells were lentivirally transduced to express eGFP.Ffluc protein for visualization (LM-NSC008 cells green, red arrows). LM-NSC008 cells highlighted and quantified within boxes 1-7 (pseudo-object was drawn at TBI site A*).

FIGS. 9A-9D. TBI-specific distribution of intranasally delivered LM-NSC008 cells in rats (day 46). (9A) Confocal image (stitched at maximum intensity) of optically cleared coronal brain slice (1-mm thick). Rats received either a TBI or sham injury and on days 7, 9, 11, 13, 15, and 17 post-surgery were IN administered LM-NSC008 cells (1×10⁶ cells in 24 μL). On day 46, the rats were euthanized, and 1-mm brain slices were cleared using CLARITY (PACT) and imaged using confocal microscopy (9A, 9B, 9C, 9D). Coronal brain sections (S1, S2, S3, S4) are shown overlapping the TBI site (LM-NSC008 cells green, red arrows). LM-NSC008 cells highlighted in selected boxes 1-9 (pseudo-object was drawn at TBI site A*).

FIGS. 10A-10F. Quantification of distribution of LM-NSC008 cells at day 25 (R10) and day 46 (R30) (10A, 10D) Distance LM-NSC008 cells migrated from the TBI site (A* in FIGS. 8 and 9). (10B, 10E) Distance quantified in combined sections (in mm) for LM-NSC008 cells migrated after intranasal administration for R10 (FIGS. 9A-9D, 4A-4H, 5A-5I, 12A-12D) and R30 (FIG. 10A-10F, 4A-4H, 5A-5I, 12A-12D, S4). (10C, 10F). Graphs represents migration dynamics of LM-NSC008 cells either from TBI site or from the brain tissue boundary quantified at days 25 (blue curve) and 46 (orange curve). Y-axis values are in millimeters (mm). Note increased number of cells close to the TBI site and minimal migration at day 25 but fewer cells at the primary TBI site and greater migration at day 46. The data show that LM-NSC008 cells are migrating throughout the brain and into regions mediating the behaviors impacted by TBI, particularly during the later time which correlates with cognitive testing.

FIGS. 11A-11D. Immunohistochemistry staining using anti human Nestin-specific antibodies to visualize LM-NSC008 cells at TBI site on day 39 (female rats, coronal paraffin sections #64). (11A) Bright field tile image of the rat brain with CCI injury site (upper right), Scale bar 1000 μm. (11 B, 11D) Parallel coronal brain sections with LM-NSC008 cells stained with DAB brown and highlighted with red arrows, 10×. (11C) 20× magnification LM-NSC008 cells at the CCI injury site. Scale bars 100 μm and 50 μm respectively.

FIGS. 12A-12D. Immunohistochemistry staining using anti human Nestin specific antibodies to visualize LM-NSC008 cells at TBI site on day 39 (female rats, coronal paraffin sections #70). (12A) Bright field image of the CCI injury site, scale bar 1000 μm. (12B, 12C, 12D) 10× and 20× magnification of the lower left area of the CCI injury site. LM-NSC008 cells are brown and highlighted with red arrows. Scale bars 100 μm and 50 μm respectively.

FIG. 13. Beginning 1 week after TBI or Sham injury, adult male rats were IN administered LM-NSC008 cells (1×10⁶ in 24 μL; TBI+NSC or Sham+NSC) or VEH (2% human albumen serum; 24 μL; TBI+VEH or Sham+VEH) on post-surgery days 7, 9, 11, 13, 15, and 17 and then were evaluated for the acquisition of spatial learning in a well-established Morris water maze. Mean (±S.E.M.) time (s) to locate the escape platform over days 18-23. *p<0.05 vs. TBI+VEH. #p<0.05 vs. TBI+VEH and TBI+NSC. Shams did not differ from one another p>0.05.

FIG. 14 illustrates a 3-D computational prediction model of NSC migration. The left panel shows fractional anisotropy with simulated paths of undirected migration from 3 different points of origin; green olfactory bulb, red corpus collosum, blue, anterior commissure. This approach will be used to predict routes and distribution of NSCs to sites of neurodegeneration and TBI. The right panel shows a coronal view of the same model.

### DETAILED DESCRIPTION

Described herein are methods for treating traumatic brain injury (TBI). Such methods include steps of administering neural stem cells (NSCs) to a subject having a TBI and providing environmental enrichment (EE) to the subject in conjunction with or in combination with the NSC administration. The EE may be provided prior to administering the NSCs, at approximately the same time as the NSC administration, or after the administration of the NSCs.

Traumatic brain injury (TBI) is a significant health care issue that affects approximately ten million individuals worldwide each year (Hyder et al., 2007). In the United States, the incidence rate is approximately 2.8 million per year (Taylor et al., 2017a) and results in long-term neurological disabilities (Goldstein, 1990; Sosin et al., 1995; Selassie et al., 2008; Summers et al., 2009; Faul et al., 2010). Although motor function and cognition are negatively impacted, clinical and experimental studies indicate that cognitive impairments, particularly higher order functioning such as executive function, are often more pronounced and prolonged (Hamm et al., 1992, 1996, 2001; Dixon et al., 1999; Kline et al., 2001, 2002a,b, 2004a,b, 2010; Cheng et al., 2008; Horneman and Emanuelson, 2009), which adversely affects quality of life (Binder, 1986; Millis et al., 2001).

While the toll of TBI on interpersonal relationships with family, friends, and coworkers is incalculable, the economic cost to society resulting from acute medical and subsequent rehabilitative care along with the loss of productivity (due to the inability to return to work even partially) accounts for billions of dollars each year (Max et al., 1991; Selassie et al., 2008). TBI is a significant health care concern, and therefore numerous strategies, particularly pharmacological interventions, to address this issue have been implemented (for reviews see Parton et al., 2005; Kokiko et al., 2007; Wheaton et al., 2009; Garcia et al., 2011). Although these various pharmacological approaches have provided significant benefits in the laboratory, successful translation to the clinic is inconsistent (Doppenberg et al., 2004; Menon, 2009). One reason for the lack of success may be that pharmacologic strategies are hindered by blood-brain-barrier (BBB) permeability. Therefore, an approach that circumvents the BBB is needed. Neural stem cells (NSCs)—immature cells that can regenerate into any type of cell in the nervous system—and NSC-based therapies may be a feasible alternative to pharmacotherapies (or used in combination with standard of care) for improving function after TBI. Furthermore, augmenting NSC therapy with neurorehabilitation (i.e., environmental enrichment [EE]) may further improve recovery.

Failure of conventional treatments for TBI led to proposing novel stem cell-based therapies (using MSCs, iPSCs, cord blood cells and NSCs) for TBI or combinational strategies to target pathophysiology of TBI and potentially to induce functional recovery and tissue regeneration (Spurlock et al., 2017; Weston and Sun, 2018; Zhang et al., 2018). Human neural stem cells (NSCs) are attractive candidates for restoration of brain function through reconstruction (cell replacement) and repair (neuroprotection) after brain trauma. NSCs have inherent pathotropism (ability to migrate) to sites of damage in the central nervous system (CNS) and therefore can be exploited for development of cellular therapies by mechanisms such as delivering growth factors, suppressing inflammation, reducing axonal injury, and/or differentiating into mature neural-lineage brain cells. In addition, NSCs have been engineered to deliver a variety of anti-cancer agents and have shown therapeutic efficacy in preclinical models of several types of primary and metastatic brain tumors (Gutova et al., 2008, 2013a, 2015). NSCs are currently being evaluated in preclinical studies and clinical trials for repair of damaged neural tissue associated with stroke, multiple sclerosis and other neurodegenerative diseases (Marchetto et al. 2010a,b; Feng and Gao 2012). Stem cell-based therapies alone or in combination with other agents has been widely investigated and well-reported in numerous disease models and recently in trauma studies (Zibara et al., 2019). Despite some success, a major obstacle to feasibility and efficacy of NSC-based therapy is ensuring sufficient numbers of viable cells reach the diseased or damaged areas in the CNS to carry out therapeutic action. To accomplish this, delivery of NSCs to the CNS has been studied by intravenous (IV), intracranial (IC), and intranasal administration (Aboody et al., 2013; Balyasnikova et al., 2013, 2014; Gutova et al., 2015, 2017). Although IV injected NSCs can cross the BBB and localize to damaged tissue, they show limited accumulation in the brain (less than 1% of injected NSCs) (Loebinger et al., 2009). Furthermore, IV administration of NSCs can lead to immune responses and other systemic complications. IC administration of NSCs avoids potential systemic reactions and results in engraftment of approximately 5-15% of injected cells (Barish et al., 2017). However, it is invasive, costly, and potentially damaging to normal tissue, and places patients at greater risk. Intranasal administration is relatively less invasive. The data (See Example 2) show a substantial number of cells at the site of injury and part of the approach is to determine precisely how many reach the site.

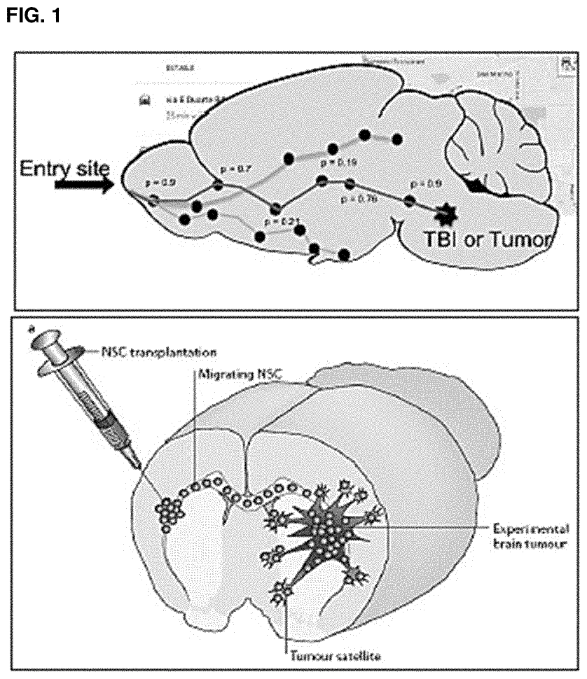

In a preclinical model of TBI, Peruzzaro and colleagues (2013) provided murine cortical eSCs alone or in combination with EE and assessed motor and cognitive outcome following bilateral prefrontal CCI injury of moderate severity in male rats. EE was initiated immediately after TBI and continued for over a month. eSCs or media were implanted 1-week post-injury, such that rats were exposed to EE both before and following transplantation. While the combination of eSCs and EE led to performance approaching sham control levels, suggesting that a certain degree of neuroprotection was achieved, there were no statistical differences in any endpoint measure relative to the monotherapies. No stringent quantitative analyses for eSC survival, migration, or differentiation into neural or glial cells were performed, hence the direct functional relevance of eSC implantation combined with EE remains to be elucidated. The absence of determining cell migration prevents the ability to correlate the behavioral outcomes with cells at the site of injury. Determining cell migration is one of the major goals of these studies as well as determining if the cells can improve motor and cognitive function (FIG. 1)

Nudi and colleagues (2015) assessed the effects of progesterone, cortical embryonic stem cells (eSCs; ˜100,000 in 2.5 μL of media i.v., 7 days post-injury), and EE alone or in two and three therapy combinations on functional outcome and the survival and differentiation of eSCs in the brain after a CCI injury of moderate severity in adult male rats. The authors reported that all therapies produced benefit over the untreated vehicle controls. Moreover, the combination therapies were more efficacious than monotherapies and the addition of EE further promoted benefits in some endpoint assessments. However, the experimental design was not rigorous and instead was extremely complicated with 10 group comparisons. Furthermore, the statistical analyses were not sufficiently stringent to prevent Type 1 errors as they used the Fisher's test rather than a more stringent test, like the Bonferroni, which corrects for multiple comparisons. Because of the inappropriate post-hoc test for the numerous comparisons, it is difficult to assess the reported outcomes. However, the rotarod data showed a significant deficit even as far out as 22 days after TBI, which in addition to the data further supports the use of that motor test in these studies.

Mahmood and colleagues (2008) conducted a study evaluating the effects of MSCs (doses of 1×10⁶ or 2×10⁶ i.v., 7 days post-injury) and simvastatin (0.5 mg/kg or 1.0 mg/kg, p.o. starting 24 h post-injury for 14 days thereafter), alone and in combination, on functional outcome in female rats after a moderate CCI. The data showed that both doses of MSCs and simvastatin, as well as all combinations, significantly improved neurological severity scores vs. TBI controls. However, the lower dose of simvastatin combined with the higher dose of MSCs revealed a synergistic effect vs. all monotherapies in improving outcome. This finding of a subtherapeutic dose of a therapy combined with stem cells supports the design of using sub-therapeutic EE (see next section) in combination with NSC. Moreover, the doses used in the current study are like two of the doses that were proposed to be used in the approach discussed herein. A limitation for translation in the current study was the route of administration (i.v) as mentioned previously only limited numbers of cells reach the site of injury.

EE is a non-invasive intervention strategy that consists of providing rats a milieu that is conducive for engaging in sensory stimulation and physical exercise in an expansive social environment (Kline et al., 2007; Sozda et al., 2010; and comprehensive reviews, Bondi et al., 2014a, 2015) that may be akin to multi-modal clinical neurorehabilitation. Basic science evidence for the benefits of a multi-modal rehabilitation setting is derived from a study where this concept was evaluated by removing one of the components of EE (i.e., exploratory, sensory, and social) and keeping the other two; this was done until all permutations were included. The data showed that while some benefit is seen when one of the components is missing, the optimal effect is seen when all are together (Sozda et al., 2010). EE may be viewed as a preclinical model of neurorehabilitation because of its ability to manifest motor and cognitive improvements after TBI as well as attenuating histological damage. Moreover, after TBI, patients may receive therapy in an inpatient setting or in a skilled nursing facility and the cognitive and physical stimulation provided by the multidisciplinary therapy can be viewed as enriching. EE has been studied in various experimental conditions. In non-TBI conditions, EE has been shown to produce numerous anatomical and physiological responses such as increased brain weight and dendritic arborization, cortical and hippocampal synaptogenesis (Frick and Fernandez, 2003; van Praag et al., 2000), and increased 5-HT1A receptor mRNA expression (Rasmuson et al., 1998). In TBI models, EE has been shown to produce alterations in DA neurotransmission (Wagner et al., 2005), increase cortical thickness (Giza et al., 2005), and increase trophic factor expression (Chen et al, 2005). In addition to plasticity-associated adaptations, which may be considered reparative mechanisms, EE also reduces lesion size after TBI (Passineau et al., 2001; Sozda et al., 2010; Monaco et al., 2013), attenuates hippocampal CA1/3 cell death (Kline et al., 2010; Sozda et al., 2010; Monaco et al., 2013), and reduces TBI-induced choline acetyltransferase-positive cell loss in medial septal neurons at 3 weeks (Kline et al., 2010), indicating it also promotes neuroprotective mechanisms. Furthermore, EE improves behavioral performance and sensory neglect after brain trauma in both adult (Held et al., 1895; Gentile et al., 1987; Rose et al., 1987) and developing rats (Giza et al., 2005) and improves motor and cognition after TBI. For example, early and continuous EE for 11 or 15 days after fluid percussion brain injury or 18 days after CCI injury has been reported to significantly improve water maze performance vs. standard (STD)-housing (Hamm et al., 1996; Passineau et al., 2001; Hicks et al., 2002; Kline et al., 2007; Hoffman et al., 2008; Sozda et al., 2010; Monaco et al., 2013). Importantly for Aim 3 of this application a 4-hr EE paradigm is not effective in either males or females when used as the sole therapy (de Witt et al., 2011; Radabaugh et al., 2016). However, it is a clinically relevant model as patients often receive ≤4-hr of rehabilitation daily (Blackerby 1990; Shiel et al., 2001; Zhu et al., 2007; Vanderploeg et al., 2008; Garcia et al., 2011). Also, because the goal of Aim 3 is to determine the effect of combining EE with NSC therapy, providing EE at longer durations (e.g., 6-hr or 24-hr) will ultimately show EE to be like, or superior, to the intended therapy as shown with pharmacological agents such as buspirone, 8-OH-DPAT, and methylphenidate (Kline et al., 2007, 2010; Bondi et al., 2014a,b; de la Tremblaye et al., 2017a, 2019; Leary et al., 2017). These combination studies were unable to detect an additive effect as increased time periods of EE were quite robust. Hence, a subtherapeutic dose of EE will afford the opportunity to determine the effects of NSC therapy, but also to determine and confirm if there is a synergistic effect to show that the combination is better than NSC treatment alone. Moreover, 6-hr and 24-hr of EE per day is not clinically relevant (Bondi et al., 2014a, 2015).

Because of the barriers to efficient NSC administration, there is an urgent need for new ways to administer NSCs that are non-invasive, cost-effective, allow efficient engraftment, and can be used for repeated treatments. To meet this need, intranasal administration of therapeutic human NSCs to sites of brain injury is optimized in a preclinical rat model and to determine the safety/efficacy and translational potential of this approach.

Specifically, an NSC-mediated therapy will be developed using an immortalized NSC cell line that stably expresses the L-MYC gene (LM-NSC008) for the treatment of TBI in adult male and normal cycling female rats after controlled cortical impact (CCI) injury. Optimal conditions should be achieved with NSC-mediated therapy including efficient and controlled cell proliferation, migration and differentiation, with a reduced risk of host immune response and lack of tumorigenicity. Furthermore, a combinational approach of LM-NSC008 cells with EE and to develop a computational model of NSC migration, fate and therapeutic efficacy of LM-NSC008 cells at TBI sites after intranasal administration will be developed. Successful translation of intranasal administration of NSCs will be more cost effective than current approaches because it can be done in an outpatient setting and can overcome the risks and hindrances noted above.

NSCs have been shown to migrate to areas of damage in the CNS. In earlier studies, well-characterized allogeneic human NSCs genetically modified to express the human L-Myc gene (LM-NSC008) migrated to and distributed at areas of damaged brain tissue (see Example 1 and Li et al., 2016). In addition, it was found that spatial learning in a well-established water maze task was also improved in the NSC group vs. the VEH-treated TBI controls. These findings lend support for the use of NSCs as proposed here to significantly enhance motor and cognitive function after TBI. Furthermore, combine NSC therapy with EE may further enhance recovery as it was shown when EE is combined with other therapies.

The studies described in the working examples below are designed to put forth a therapeutic approach that circumvents the BBB such that the therapy (NSC) can migrate and accumulate at the site of injury in sufficient quantity to produce motor and cognitive benefits after a TBI of moderate severity in adult male and normal cycling female rats. Additionally, the studies will have a significant translational impact by providing a route of administration (intranasal) that is less invasive than other routes of stem cell administration. Furthermore, the preclinical model of neurorehabilitation mimics the “real world” based on timing of initiation and amount of exposure as patients typically receive about 4-hr of EE daily and not immediately after TBI. It is believed that the experiments and predicted findings have the potential to instill confidence in the use of NSC therapy and physiotherapy (i.e., EE) that could ultimately serve as a viable treatment for the millions of TBI survivors who currently have little options for treatment of their long-term and debilitating motor and cognitive faculties.

The studies below are also the first known to develop and evaluate the therapeutic efficacy of the NSC cell line LM-NSCOO8s for the treatment of TBI in a model of controlled cortical impact injury in both male and female rats. The proposed studies are also the first to investigate the combination of LM-NSC008 cells with a preclinical model of neurorehabilitation that is clinically relevant. Further, CLARITY and three-dimensional (3D) confocal microscopy may be developed and used to interrogate and predict NSC migration in the brain. This approach will provide a much-needed efficient method (both in terms of time and cost) to measure the numbers of viable NSCs that reach sites of TBI in 3D. The use of CLARITY and 3D confocal microscopy to define 3D distribution of NSCs in directed migration will guide the analysis of NSC migration and subsequent therapeutic efficacy.

As the success of stem cell-based therapies is contingent on efficient cell delivery to damaged areas, neural stem cells (NSCs) have promising therapeutic potential because they inherently migrate to sites of central nervous system (CNS) damage.

According to some embodiments and to explore the possibility of NSC-based therapy after traumatic brain injury (TBI), isoflurane-anesthetized adult male rats received a controlled cortical impact of moderate severity (2.8 mm deformation at 4m/s) or sham injury (i.e., no cortical impact). Beginning 1-week post-injury, the rats were immunosuppressed, and 1 ×10⁶ human NSCs (LM-NS008.GFP.ffLuc) or vehicle (VEH; 2% human serum albumen) were administered by intranasal (IN) delivery on post-operative days 7, 9, 11, 13, 15, and 17. To evaluate the spatial distributions of the LM-NSC008 cells, half of the rats were euthanized on day 25, one day after completion of the cognitive task, and the other half were euthanized on day 46. 1-mm thick brain sections were optically cleared (CLARITY), and volumes were imaged by confocal microscopy. In addition, LM-NSC008 cell migration to the TBI site was observed by immunohistochemistry for human specific Nestin at day 39. Acquisition of spatial learning was assessed in a well-established Morris water maze on six successive days beginning on post-injury day 18. IN administration of LM-NSC008 cells after TBI (TBI+NSC) significantly facilitated spatial learning relative to TBI+VEH rats (p<0.05) and had no effect on Sham+NSC rats. Overall, these data indicate that IN-administered LM-NSC008 cells migrate to sites of TBI damage, and that their presence correlates with cognitive improvement. Additional studies will expand on these preliminary findings by evaluating other LM-NSC008 cell dosing paradigms and evaluating mechanisms by which LM-NSC008 cells contribute to functional recovery.

In sum, the innovation is that the proposed studies are the first to begin a comprehensive evaluation of NSC and NSC+EE in the pursuit of a therapeutic strategy that could revolutionize treatments for TBI patients by providing a method (intranasal) for non-invasive, repeat treatments that can circumvent the BBB, which impedes pharmacotherapies from entering the CNS in sufficient quantities to be viable. Hence, the proposed studies are also innovative in the comparative models used and the translational potential to humans.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Accordingly, the invention is not limited except as by the appended claims. Further, all references cited in the disclosure are hereby incorporated by reference in their entirety, as if fully set forth herein.

### EXAMPLES

Traumatic brain injury (TBI) affects more than 10 million individuals worldwide each year and is a major cause of disability, resulting in long-term functional deficits for TBI survivors. To date, the major unmet needs for treating TBI are effective strategies to restore neuronal networks and recover function. Although pharmacologic strategies are the most common approach to treat TBI, they, and other approaches, are hindered by blood-brain-barrier (BBB) permeability. Therefore, a therapeutic approach that circumvents the BBB is needed. Neural stem cell (NSC)-based therapies may be a feasible alternative to pharmacotherapies for improving function after TBI. However, stem cell-based therapies are contingent on efficient delivery to the areas of damage. Well-characterized allogeneic human NSCs genetically modified to express the human L-Myc gene (LM-NSC008) migrated to and distributed at damaged brain regions, and rats showed improved spatial learning after receiving intranasally delivered NSCs. The studies provided herein are designed to determine and confirm that 1) intranasally-delivered LM-NSC008 NSCs will migrate to TBI sites, accumulate in sufficient quantities, and contribute to motor and cognitive recovery post-injury, and 2) augmenting NSCs with environmental enrichment (EE) will provide further benefits.

### Example 1

### Intranasally administered L-Myc Immortalized Human Neural Stem Cells Migrate to 1 Primary and Distal Sites of Damage after Cortical Impact and Enhance Spatial Learning

As the success of stem cell-based therapies is contingent on efficient cell delivery to damaged areas, neural stem cells (NSCs) have promising therapeutic potential because they inherently migrate to sites of central nervous system (CNS) damage. To explore the possibility of NSC-based therapy after traumatic brain injury (TBI), isoflurane-anesthetized adult male rats received a controlled cortical impact of moderate severity (2.8 mm deformation at 4m/s) or sham injury (i.e., no cortical impact). Beginning 1-week post-injury, the rats were immunosuppressed, and 1 ×10⁶ human NSCs (LM-NS008.GFP.ffLuc) or vehicle (VEH; 2% human serum albumen) were administered by intranasal (IN) delivery on post-operative days 7, 9, 11, 13, 15, and 17. To evaluate the spatial distributions of the LM-NSC008 cells, half of the rats were euthanized on day 25, one day after completion of the cognitive task, and the other half were euthanized on day 46. 1 mm thick brain sections were optically cleared (CLARITY), and volumes were imaged by confocal microscopy. In addition, it was observed that LM-NSC008 cells migrated to the TBI site by immunohistochemistry for human specific Nestin at day 39. Acquisition of spatial learning was assessed in a well-established Morris water maze on six successive days beginning on post-injury day 18. IN administration of LM-NSC008 cells after TBI (TBI+NSC) significantly facilitated spatial learning relative to TBI+VEH rats (p<0.05) and had no effect on Sham+NSC rats. Overall, these data indicate that IN-administered LM-NSC008 cells migrate to sites of TBI damage, and that their presence correlates with cognitive improvement. Additional studies will evaluate other LM-NSC008 cell dosing paradigms and evaluating mechanisms by which LM-NSC008 cells contribute to functional recovery.

Introduction

The current study was conducted to determine, using a well-established rat model of TBI (Kline et al. 2001; Dixon et al. 1991; Bondi et al. 2014c; Bondi et al. 2014b), whether IN administered L-MYC-immortalized human NSCs (LM-NSC008 cells) accumulate in damaged brain tissue and facilitate cognitive recovery. To accomplish this purpose, seven doses of LM-NSC008 cells were administered once every other day beginning 1 week after controlled cortical impact (CCI) or sham injury to adult rats, and acquisition of spatial learning as well as patterns of early and late NSC migration were assessed. The findings indicate that IN delivered NSCs migrate to primary and distant TBI sites and facilitate acquisition of spatial learning. As NSC-based therapy can be delivered IN, it is a potentially feasible alternative to pharmacotherapies for cognitive recovery after TBI. If this therapeutic approach can be successfully translated to the clinic, it would allow for extended, cost-effective, and relatively non-invasive treatments for TBI patients.

Materials and Methods

Subjects. Forty-eight adult male Sprague-Dawley rats (Envigo, Indianapolis, IN) weighing 300-350 g on the day of surgery were housed in pairs in Plexiglas cages with ad libitum food and water and maintained in a temperature (21±1° C.) and light (on 0700 to 1900) controlled environment. After a week of acclimatization, the rats were randomly assigned to one of four experimental groups: TBI+NSC (n=17); TBI+VEH (human serum albumin; n=16); Sham+NSC (n=6) and Sham+VEH (n=9). An additional 10 adult female rats weighing 250-275 g on the day of surgery were included for IHC staining using anti human Nestin-specific antibodies to visualize LM-NSC008 cells at the TBI site on day 39; TBI+NSC (n=3); TBI+VEH (n=3); Sham+NSC (n=2) and Sham+VEH (n=2). Procedures for TBI and Sham surgeries, IN administrations, and cognitive assessments were approved by the University of Pittsburgh's Institutional Animal Care and Use Committee and the Institutional Biosafety Committee.

Surgery. A controlled cortical impact (CCI) injury was produced as previously described (Kline et al. 2001; Kline et al. 2007; Kline et al. 2010; Dixon et al. 1991; Bondi et al. 2014a; Kline et al. 2002b; Lajud et el. 2019). Briefly, rats were fully anesthetized with inspired isoflurane (4% induction and 2%maintenance with a 2:1 ratio of N2O:O2), intubated, fixed in a stereotaxic frame, and ventilated mechanically. Adhering to aseptic technique, a midline scalp incision was made to expose the skull, and a craniectomy was made in the right hemisphere between bregma and lambda and the sagittal and coronal sutures with a high-speed dental drill. The bone flap was removed, and the surgical opening was enlarged further to ensure clearance of the impact tip (6 mm, flat), which was centered and lowered through the craniectomy until it touched the dura mater. The rod was then retracted, and the impact tip was advanced 2.8 mm to produce a moderate brain injury (2.8 mm tissue deformation at 4 m/s). Following impact, the rats were sutured, extubated, assessed for acute neurological function (flexion and righting reflexes), and allowed to recover spontaneous ambulation prior to being returned to their home cage. Core temperature was maintained at 37° C.±0.5° C. with a heating blanket. Sham controls underwent all procedures except for the CCI.

Acute neurological evaluation. Hind limb reflexive ability was assessed after the cessation of anesthesia and removal from the stereotaxic apparatus by gently squeezing the rats' hind paws every 5 s and recording the time to elicit a withdrawal response. Return of the righting reflex was determined by the time required to turn from the supine to prone position three consecutive times. These neurological indices are sensitive determinants of injury severity (Kline et al. 2001; Kline et al. 2007; Kline et al. 2010; Lajud et el. 2019; Kline et al. 2002a; Bondi et al. 2015).

Administration of LM-NSC008 cells or VEH. Beginning 1 week after TBI or sham surgery, the rats were IN administered 1 ×10⁶ human NSCs (LM-NS008) in 24 μL of vehicle (VEH; 2% human serum albumin) or VEH alone on post-operative days 7, 9, 11, 13, 15, and 17. The administration procedure was as previously reported, and shown to achieve distribution and efficacy of cellular therapy (Balyasnikova et al., 2014). Subcutaneous injections of Cyclosporin A (10 mg/kg; Alfa Aesar, Haverhill, Mass.) were administered daily to all rats beginning on post-operative day 5 for the duration of the study.

Cognitive function: acquisition of spatial learning and memory retention. Acquisition of spatial learning was assessed using a Morris water maze (MWM) task that has been shown to be sensitive to learning deficits after TBI (Sozda et al. 2010; de Witt et al. 2011; Monaco et al. 2013; Radabaugh et al. 2016; Bleimeister et al. 2019). Training was conducted every day on post-operative days 18 through 23. Briefly, the maze (180 cm diameter plastic pool) was filled with water (26±1° C.) to a depth of 28 cm and located in a room with visual cues saliently displayed on the walls. The escape platform was a clear Plexiglas stand (10 cm diameter) placed 2 cm below the surface of the water and positioned 26 cm from the edge of the pool in the southwest quadrant for all trials. Learning acquisition consisted of four trials, with random placement into the pool from one of each cardinal direction (north, east, south, and west). Each rat was given a maximum of 120 s on each trial to find the submerged platform. Rats that failed to find the platform in the allotted time were manually guided to and placed on the platform, where they remained for 30 s before being removed and placed in a warming chamber (ThermoCare®, Paso Robles, Calif.) for a 4-min inter-trial interval. The mean of the 4 daily trials for each rat were used in the statistical analyses.

On post-surgery day 24, a single probe trial was provided to evaluate memory retention. Briefly, during the probe trial, the platform was removed from the pool, and the rat was placed in the maze at the location point most distal to the southwest quadrant where the platform was previously located (i.e., target quadrant) and allowed to freely explore the pool for 30 s. The percent time spent in the target quadrant was recorded and used in the statistical analysis. Lastly, after the probe trial, the platform was raised 2 cm above the water surface and white tape was wrapped around the rim making it visible to the rats, which controls for visual acuity, and non-spatial contributions like sensorimotor performance and motivation. The ANY-Maze software and tracking system (Stoelting, Wood Dale, Ill.) was used to automatically record all data, which included time to locate the platform, percent time in the target quadrant, and swim speed.

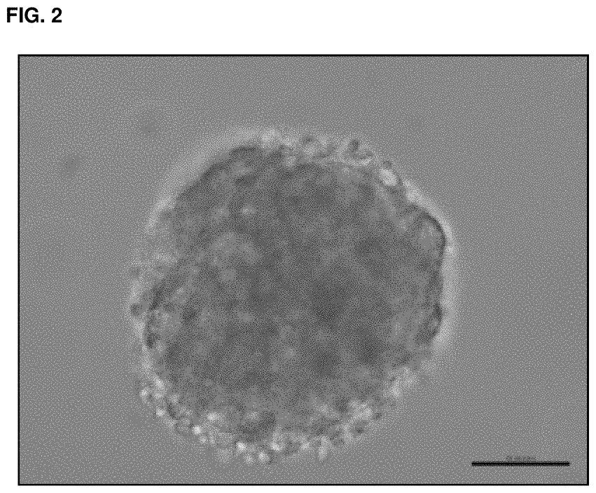

Generation and characterization of primary NSC cultures. Isolation and propagation of fetal brain neural stem cells and immortalization with L-MYC gene has been previously described (Li et al. 2016; Rockne et al. 2018). LM-NSC008 cells were cultured in serum-free NSC medium (RHB-A medium; Stem Cell Science) supplemented with 10 ng/mL basic fibroblast growth factor (bFGF), 10 ng/mL epidermal growth factor (EGF), 2 mM L-glutamine (Invitrogen), Gem21 NeuroPlex Serum-Free Supplement (GeminiBio-Products, #400-160). bFGF and EGF were added every other day and the media were completely changed every 7 days. FIG. 2. LM-NSC008 cells were modified using lentivirus to express enhanced green fluorescent protein (eGFP) and ffLuc (LM-NSC008.GFP.ffLuc) for visualization in vivo and in brain sections by fluorescent microscopy.

Expansion of LM-NSC008 cells to create master cell banks. Generation and characterization of LM-NSC008 cells stably expressing L-MYC has been previously described (Li et al. 2016). Cells were passaged and characterized for up to passage 50 (Rockne et al. 2018). Briefly, LM-NSC008 cells were expanded to master cell banks using the Quantum Cell Expansion system (Terumo BCT), and frozen at a concentration of 2.4×107 cells in CryoStore (BioLife Solutions) in liquid nitrogen. LM-NSC008 cells were thawed, washed with PBS, and administered via IN drops into rats bearing CCI or Sham injury using previously optimized protocols (Gutova et al. 2015; Tirughana et al. 2018).

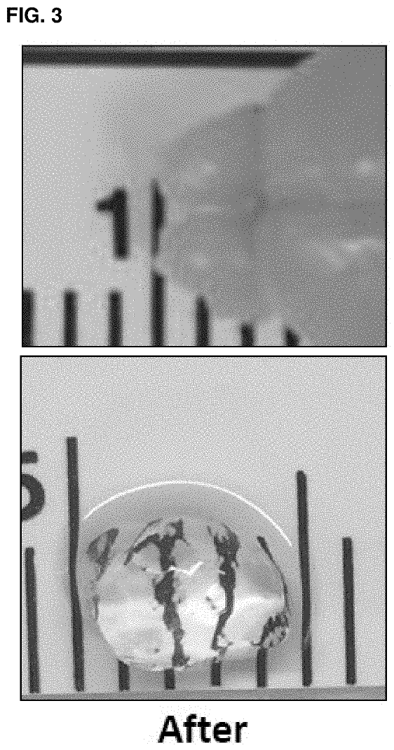

Brain tissue clearing (CLARITY) and distribution of LM-NSC008 cells. Rats received an overdose of sodium pentobarbital (i.p.) on day 25 (early time point) or day 46 (late time point) after surgery and then perfused with ice cold 0.1 M phosphate buffered saline, fixed with 4% PFA, and brains harvested. Brains were prepared as thick (1 mm) sections using acrylamide-based tissue clearing (CLARITY) (Treweek et al. 2015; Yang et al. 2014). FIG. 3. Brain slices were then imaged by confocal microscopy (z-axis resolution) and examined for the presence of the LM-NSC008 cells by fluorescent microscopy.

For IHC, paraffin-embedded brain sections were sectioned into 10 pm coronal sections and stained (every 5th section) with anti-human Nestin antibody (Millipore; Cat# MAB5326, at 1:200 dilution) by the City of Hope Anatomic Pathology Core. Brain sections were then processed for antigen retrieval with Proteinase K (Dako ready-to-use Cat # S3020), incubated in peroxidase quenching solution (0.3% H2O2 made in 100% methanol for 20 min at room temperature), and then in blocking solution (50% BlockAid, Invitrogen B10710; 50% Western Blocking Reagent, Roche Applied Sciences 11921673001; 1% Triton-100x). Sections were then stained with primary antibody in blocking solution and incubated overnight at 4° C., followed by several washes in PBS and reaction with biotinylated secondary antibody (1:250 dilution, Vector BA-2001) for 1 h as previously described (Rockne et al., 2018). Sections were washed, incubated in avidin-biotin complex (ABC) solution for 1 h at room temperature and 5 min in 3, 3′-Diaminobenzidine (DAB) substrate solution containing 0.25% H2O2. Finally, the brain sections were washed and mounted with Cytoseal 8 mounting media (Richard-Allan Scientific) and imaged.

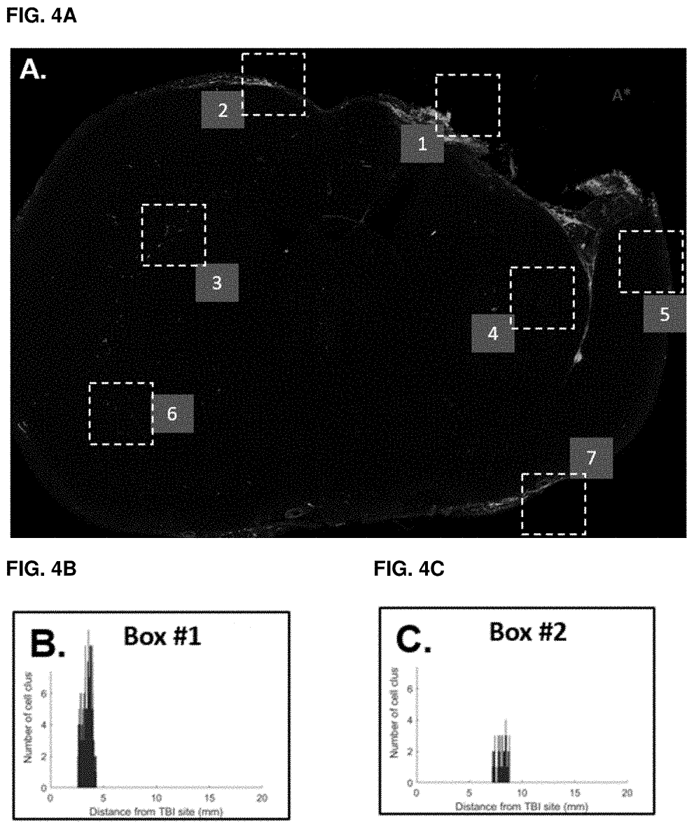

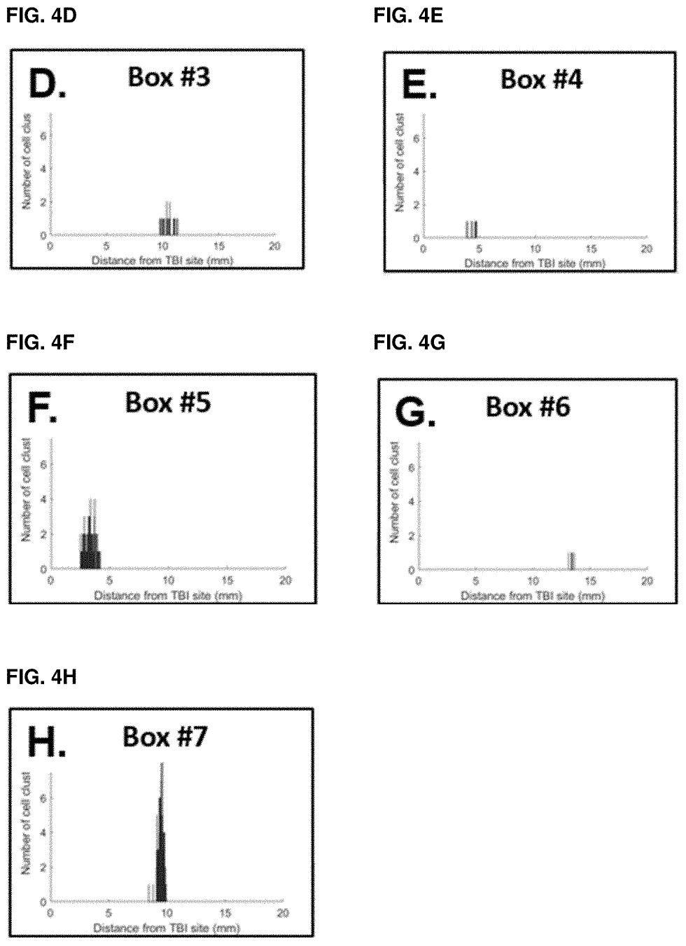

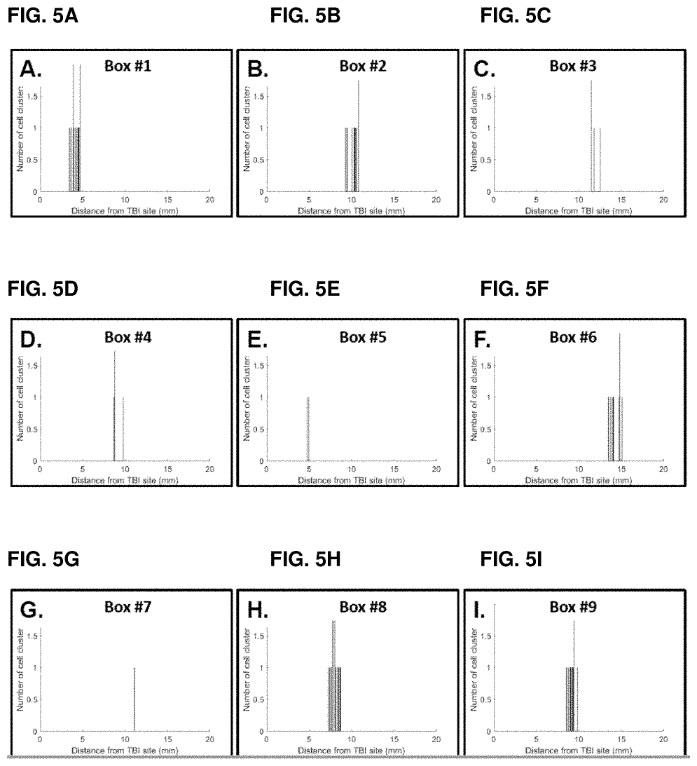

Clarity image analysis. Maximum intensity projections of optically-sectioned brain sections were generated using ImageJ (Schneider et al., 2012). Background subtraction was performed with a 50-pixel radius. The resulting images were manually thresholded to generate the distribution of NSCs in the imaged tissue section. A mask of the tissue section was created using the maximum intensity projection of the entire brain. Segmented NSCs within the tissue mask were analyzed in MATLAB (Mathworks, MA). Location of TBI centroid was manually input and the distance of each pixel identified as a part of an NSC from the TBI centroid was calculated. Additionally, based on the tissue mask, the distance of each of these pixels to the nearest edge of the tissue mask was calculated. The distribution of these pixels from the TBI site and the edge of the tissue is presented as histograms and cumulative distribution functions. Subsections of the images demonstrating NSC presence were analyzed separately to evaluate the distance of NSCs from the TBI site (FIGS. 4A-4H, 5A-5I). For this purpose, instead of each pixel, clusters of pixels connected to each other were evaluated for distance to the TBI site.

Statistical analyses. All behavioral analyses were performed using Statview 5.0.1 (Abacus Concepts, Inc., Berkeley, Calif.) on data collected by blinded experimenters. Assessment of spatial learning was conducted with repeated-measures analysis of variance (ANOVA). Probe trial, visible platform, and swim speed were analyzed using one-factor ANOVAs, as were the acute neurological outcomes (i.e., hind limb withdrawal reflex times and righting reflex times). When the overall ANOVA revealed significant effects, the Newman-Keuls post-hoc test was used to determine specific group differences. The results are expressed as the mean ±standard error of the mean (S.E.M.) and are considered significant when p 0.05. Eight rats (males) died post-surgery and thus the statistical analyses were performed on the data from 40 rats that made up the final composition of groups: TBI+NSC (n=15), TBI+VEH (n=14), Sham+NSC (n=6), Sham+VEH (n=5).

Results

Acute neurological function. No differences were observed between the groups randomized to TBI+NSC and TBI+VEH in hind limb withdrawal reflex after a brief paw pinch or for return of the righting reflex following the cessation of anesthesia (p>0.05; Table 1). The lack of significant differences with these acute neurological indices suggests that both TBI groups experienced an equivalent level of injury. Additionally, no differences were observed between the Sham+NSC and Sham+VEH rats on either acute neurological outcome (p>0.05).

TABLE 1
Reflex behavior (s)

| | | TBI + LM-NSC008 | TBI + VEH | |
|---|---|---|---|---|
| Withdrawal | Left | 167.0 ± 8.7 | 167.2 ± 4.3 | p > 0.05 |
| reflex | Right | 155.9 ± 4.9 | 162.6 ± 4.1 | p > 0.05 |
| Righting | | 389.6 ± 19.2 | 395.9 ± 19.1 | p > 0.05 |
| reflex | | | | |
| | | Sham + LM-NSC008 | Sham + VEH | |
| Withdrawal | Left | 20.8 ± 3.1 | 18.0 ± .7 | p > 0.05 |
| reflex | Right | 13.2 ± 1.2 | 16.3 ± 2.9 | p > 0.05 |
| Righting | | 127.7 ± 5.3 | 121.2 ± 6.4 | p > 0.05 |
| reflex | | | | |

Mean (±S.E.M.) acute neurological assessments. No significant differences were revealed between the TBI + LM-NSC008 and TBI + VEH groups in time (s) or between the Sham + LM-NSC008 and Sham + VEH groups to elicit a right and left hind paw reflexive withdrawal (after a brief paw pinch) or righting reflex after the cessation of anesthesia.

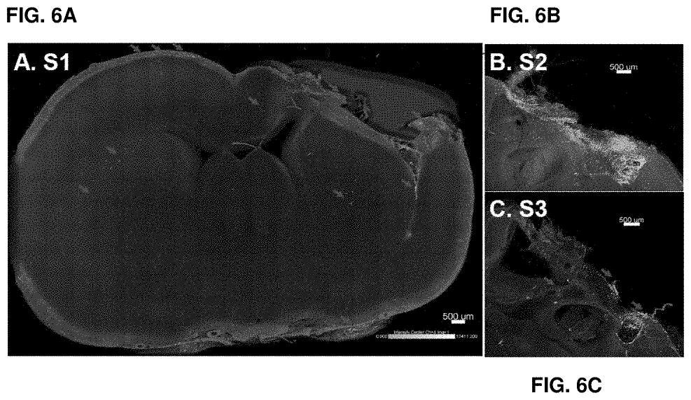

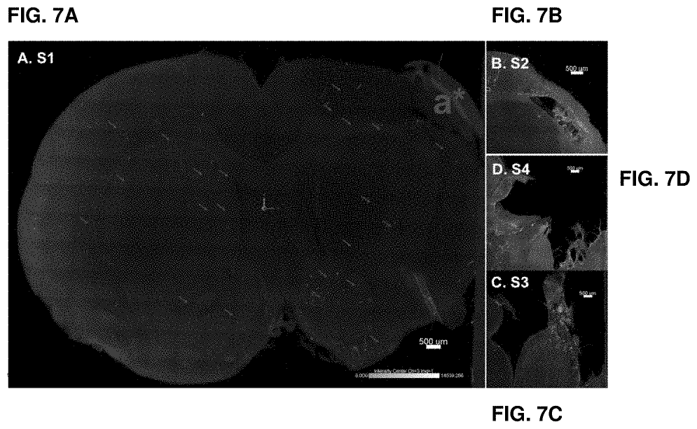

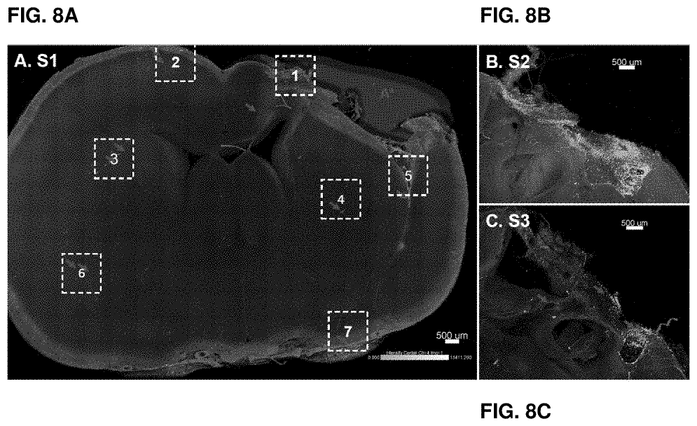

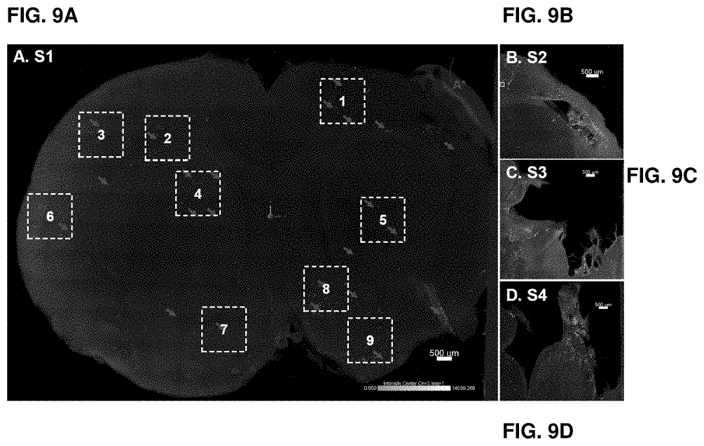

Spatial distribution of IN administered LM-NSC008 cells. Within the brain parenchyma, imaging of LM-NSC008 cells was performed using confocal 3D imaging of optically-cleared 1 mm-thick brain sections at 25 and 46 days after TBI (early and late time points, respectively; FIGS. 6A-6C and 7A-7C). Square boxes were added to images to highlight cell clusters as shown in FIGS. 8A-8C and 9A-9D. Areas of brain tissue highlighted by the square boxes in FIGS. 8A-8C and 9A-9D were used for the analysis of number of cell clusters, and the distance LM-NSC008 cells have migrated from the TBI site and from the edge of the brain tissue were analyzed. At the early time point most LM-NSC008 cells were localized at the site of TBI damage in optically-cleared sections (FIGS. 8A.S1, 8B.S2, 8C.S3) but at the later time point (day 46), LM-NSC008 cells were found at sites distant to the primary injury (FIGS. 9A.S2, 9B.S2, 9C.S3, 9D.S4), indicating that LM-NSC008 cells migrate well beyond the lesion site, possibly following damaged axonal tracts.

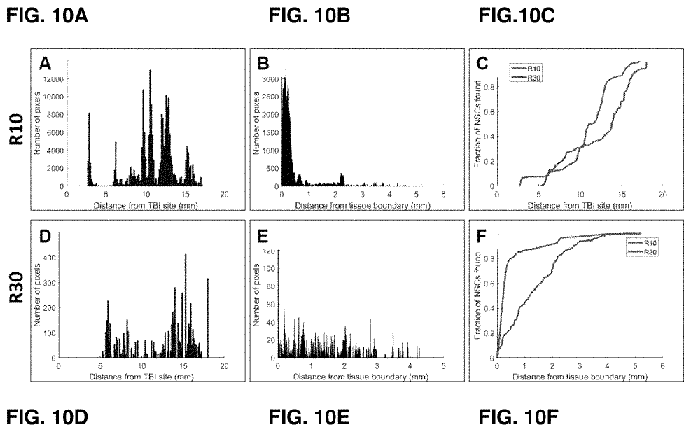

Total LM-NSC008 cell distribution in early and late timepoints are summarized from the data of R10 and R30, rats that were euthanized at days 25 and 46, respectively (FIGS. 10A-10F). Quantification of LM-NSC008 cell distributions was done by measuring distances from TBI sites and from the brain surface. The positions of the LM-NSC008 cells suggests that, conceivably, IN administered NSCs could take neuronal (olfactory), vascular or lymphatic migration routes to distribute in the subarachnoid space and then enter the brain parenchyma.

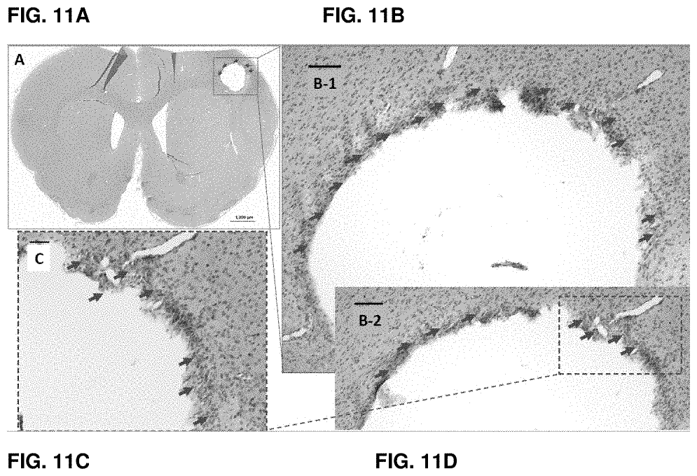

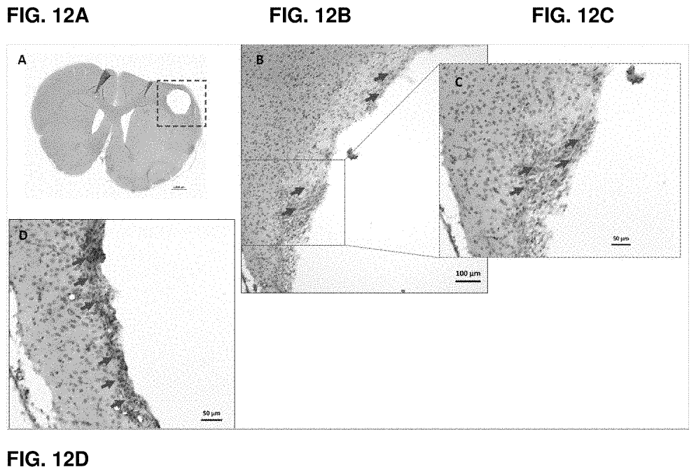

To probe for LM-NSC008 cells at the TBI site, female rat brains were harvested, fixed, sectioned, and stained for histological examination and IHC visualization of human nestin on day 39 (FIGS. 11A-11C). LM-NSC008 (human nestin-expressing) cells were found lining the edge of the CCI injury site (red arrows), at edges of the brain, and within the brain parenchyma (FIGS. 11A-11C and 12A-12D, red arrows).

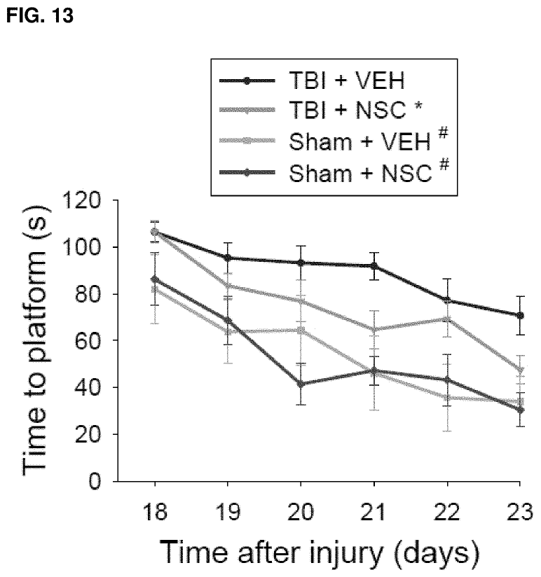

Acquisition of spatial learning. Analysis of the spatial learning data revealed significant Group (F3,36 =9.37, p =0.0001) and Day (F5,180 =18.07, p<0.0001) differences. The TBI+NSC group learned the location of the submerged escape platform (i.e., acquired spatial learning) significantly better than the TBI+VEH group (p<0.05). Time to locate the visible platform was also significantly longer for the TBI+VEH group relative to the Sham+NSC and Sham+VEH groups (p<0.05). Post-hoc analysis revealed no differences between the Sham+NSC and Sham+VEH groups (p >0.05), and both were better than the TBI+NSC and TBI+VEH groups (p<0.05; FIG. 13). Swim speed did not differ among the groups (range=33.6±1.7 cm/s to 34.8±2.0 cm/s; p>0.05). Also, no differences were observed among the groups in percent time spent in the target quadrant during the probe trial (p>0.05).

Discussion

These data indicate that IN administration of human LM-NSC008 cells after TBI significantly improves acquisition of spatial learning relative to VEH-treated TBI rats, and that LM-NSC008 cells distribute throughout damaged brain tissue and into normal brain regions. That IN administered LM-NSC008 cells were present in sufficient numbers to improve cognition is a significant finding, as a major obstacle to the feasibility and efficacy of NSC-based therapy is ensuring that sufficient numbers of viable cells reach the diseased or damaged areas in the CNS (Balyasnikova et al. 2014; Aboody et al. 2013; Gutova et al. 2015; Li et al. 2016). Studies have shown that although IV-administered NSCs can cross the blood brain barrier and localize to damaged tissue, they show limited accumulation in the brain (less than 1% of injected NSCs) (Loebinger et al. 2009). IV administration of NSCs can also lead to systemic complications. Intracranial administration of NSCs, while avoiding potential systemic reactions and resulting in engraftment of approximately 5-15% of injected cells (Barish et al. 2017), is invasive, costly, and potentially damaging to normal tissue.

Other stem cell-based therapies investigated for the treatment of TBI include use of NSCs derived from bone-marrow-derived mesenchymal stem cells, embryonic stem cells or from umbilical cord-derived mesenchymal stem cells (Lois & Alvarez-Buylla 1993; Gage 1994; Gage 2003; Doetsch et al. 1997; Chirumamilla et al. 2002; Rice et al. 2007; Mahmood et al. 2008; Peruzzaro et al. 2013; Nudi et al. 2015; Rolfe & Sun 2015; Kline et al. 2016; Zibara et al. 2019). Unlike LM-NSC008 cells that showed a cognitive benefit when administered alone after TBI, other NSC-based therapies produced a significant behavioral benefit only when combined with environmental enrichment (EE) in a rehabilitative strategy (Peruzzaro et al. 2013; Nudi et al. 2015; Duncan et al. 2015). Based on previous combination therapies (Kline et al. 2016) and the efficacy of LM-NSC008 cells in the current study, combining LM-NSC008 cells with EE after TBI may work in synergy or additively to produce benefits even greater than that of LM-NSC008 cells alone.

Also, in rodent models, endogenous NSCs can generate new neurons and glial cells in regions of adult neurogenesis, such as the subventricular zone (SVZ) and dentate gyrus (DG) of the hippocampus (Lois & Alvarez-Buylla 1993; Doetsch et al. 1997; Gage & Temple 2013). Post-TBI stem cell recruitment to the SVZ and DG indicate inherent attempts of the brain to repair and regenerate after injury, which might be further mediated by administration of exogenous LM-NSC008 cells (Chirumamilla et al. 2002; Rice et al. 2007). Endogenous neurogenesis can be enhanced by introduction of exogenous growth factors, VEGF, statins, and progesterone, but stem cell therapies may more effectively enhance endogenous neurogenesis by integrating into host tissue as well as providing trophic support (Mahmood et al. 2008; Kline et al. 2016; Najbauer et al. 2012). Gennai and others have demonstrated that stem cells and progenitor can migrate to the injured brain regions and proliferate, exerting protective effects through possible cell replacement and release of anti-inflammatory and growth factors in pre-clinical studies (Gennai et al. 2015). While stem/progenitor therapies demonstrated improvement after TBI and stroke in pre-clinical and clinical studies, exact mechanism of the recovery is not known. Some studies reported a limited migration ability in vivo, however, the benefits of cell-based therapy have been clearly demonstrated (Haus et al. 2016). Whether brain repair occur via cell replacement, immunomodulation or endogenous tissue repair mechanisms, questions which can be further investigated (Cox et al. 2018).

By visualizing LN-NSC008 cells in situ, behavioral outcomes could be correlated with the presence of NSCs at injury sites. Future studies should expand on these findings by evaluating strategies to enhance LM-NSC008 cell accumulation, including dosing paradigms. While not evaluated in this study, previous work has demonstrated that LM-NSC008 cells are not tumorigenic in vivo (tested for up to 12 months in immunodeficient mice), suggesting that they will be safe to use as a therapy for TBI (Rockne et al. 2018). Regarding possible sex differences, preliminary data shows that the benefits of LM-NSC008 cell therapy are equivalent in immunosuppressed female rats after TBI (unpublished).

Conclusions

These findings indicate that IN administration resulted in distribution of LM-NSC008 cells at TBI and distant to TBI sites in a rat model of CCI. LM-NSC008 cell distribution leads to improved recovery of cognitive performance, thereby demonstrating the potential utility of IN delivery of LM-NSC008 cells in a therapeutic context. This work also demonstrates the feasibility of using tissue clearing and volume imaging as a means of evaluating LM-NSC008 cell migration and distribution in the brain. These measures will aid in optimizing dose, timing and route of NSC-mediated cellular therapies for clinical use.

### Example 2

### Determining the Optimal Dose of NSC Delivery for Maximal Distribution to areas of TBI Damage (Prophetic)

Two dosing paradigms will be tested: one of two bolus doses (6×10⁶ or 12×10⁶ NSCs) or vehicle will be given on day 7 after moderate TBI or sham injury, or a lower dose (1×10⁶ or 2×10⁶ NSCs) will be given on alternate days starting on day 7 post-surgery through day 17. Computational analytical methods applied to optically-cleared brain sections (CLARITY technique) will be used to quantify and validate NSC migration and distribution in TBI vs. shams. Characterizing NSC distribution in 3D tissue combined with route finding algorithms will allow us to predict NSC dosing-dependent routes of migration and brain tissue biodistribution.

Protocol. NSCs (LM-NSC008) were genetically modified (using lentivirus) to express green fluorescent protein (eGFP) and firefly luciferase (FFluc) genes and were used for Xenogen imaging in vivo and visualization in 2 dimensional (2D) histological sections and optically-cleared brain sections (Gutova-Rockne and preliminary results FIGS. 9A-9D and 11A-11C). LM-NSC008 cells will be prepared for intranasal administration according to established SOPs (Gutova). Briefly, LM-NSC008 cells will be thawed, washed and re-suspended in PBS at concentrations of (6×10⁶, 12×10⁶ in 24 μl or 1×10⁶, 2×10⁶ in 24 μl) for administration (on thaw) keeping the volume of intranasal drops the same. Rats will receive intranasal injections (4 μl per drop, 3 drops per nostril, with 2 min intervals, total 24 μl) of LM-NSC008 20 min after disruption of the nasal epithelium with hyaluronidase. Bolus or six alternate day doses of LM-NSC008 cells will be administered starting on day 7 post TBI or sham injury (to mimic acute phase post TBI). Computational analytical methods applied to optically-cleared brain sections (CLARITY) will be utilized to quantify and validate NSC migration and distribution in TBI vs. sham brains as described in detail in the Methods Section.

TABLE 1
Determine the optimal dose of NSC delivery for
maximal distribution to areas of TBI damage

| Groups | TBI (M) | TBI (F) | Sham (M) | Sham (F) |
|---|---|---|---|---|
| NSC (6 × 10⁶, 12 × 10⁶) | n = 6, | n = 6, | n = 6, | n = 6, |
| or VEH, day 7 | 6, 6 | 6, 6 | 6, 6 | 6, 6 |
| post-surgery | | | | |
| NSC (1 × 10⁶, 2 × 10⁶) | n = 6, | n = 6, | n = 6, | n = 6, |
| or VEH, days 7, 9, | 6, 6 | 6, 6 | 6, 6 | 6, 6 |
| 11, 13, 15, 17 | | | | |
| post-surgery | | | | |

N = 144 *Groups sizes were determined based on our published and preliminary data showing that 6 per group is sufficient to quantify distribution of NSCs to areas of TBI damage. While sham rats may not be critical for both males and females to determine NSC migration, both groups are necessary for comparison of behavioral assessments and subsequent dissemination and publication of the results.

Discussion. The NSC dose of 1×10⁶ provided once on alternate days beginning on day 7 post-surgery was used in preliminary studies and showed cell migration and spatial learning relative to VEH controls. To further determine maximal migration to the injury site a higher dose in the alternate day administration paradigm as well as single bolus doses will be used. The single bolus doses were designed to be 6× that of the once daily doses to keep the total number of NSCs consistent, but to provide an opportunity to determine the benefits of single vs. multiple administrations.

### Example 3

### Determining the Extent to which NSC Therapy Improves Motor Outcome and Cognition (Reference Memory and Executive Function) (Prophetic)

These studies are conducted to determine the extent to which NSC therapy improves motor function and cognition in TBI rats via repair and/or cell replacement (or immunomodulatory effects) of the damaged brain tissue. The optimal dose of NSCs, determined by the greatest distribution at the site of injury, from Example 2 will be administered to separate cohorts of rats at one of three times (7-d [acute phase], 21-d [delayed phase], and 3-mo [chronic phase]) after TBI or sham injury. Intranasally-delivered LM-NSC008 cells that have migrated and accumulated in sufficient quantities to be therapeutically viable will likely enhance motor and cognitive recovery post-injury.

Protocol. The dose of NSCs determined in Example 2 to be optimal will be administered to separate cohorts of rats at one of three times (7-d [acute phase], 21-d [delayed phase], and 3-mo [chronic phase]) after moderate TBI or sham injury to determine the extent to which they improve motor function (rotarod test) and cognition (MWM and attentional set-shifting; AST), as well as cell migration at acute, delayed, and chronic phases. Details regarding behavioral assessments and CLARITY are described in Example 5 below.

TABLE 2
Determine the extent to which NSC therapy improves
motor function and cognition in TBI rats via repair
and/or cell replacement of the damaged brain tissue

| Groups | TBI (M) | TBI (F) | Sham (M) | Sham (F) |
|---|---|---|---|---|
| Optimal NCS dose from | n = 10, | n = 10, | n = 10, | n = 10, |
| Ex. 2 and VEH, day 7 | 10 | 10 | 10 | 10 |
| post-surgery | | | | |
| Optimal NCS dose from | n = 10, | n = 10, | n = 10, | n = 10, |
| Ex. 2 and VEH, day 21 | 10 | 10 | 10 | 10 |
| post-surgery | | | | |
| Optimal NCS dose from | n = 10, | n = 10, | n = 10, | n = 10, |
| Ex. 2 and VEH, day 90 | 10 | 10 | 10 | 10 |
| post-surgery | | | | |

N = 240 * Groups sizes were determined based on our published behavioral studies showing that 10 rats per group are required to provide sufficient power (typically ranging from .974 to 1) for behavioral assessments. All rats will be in standard (STD) housing conditions for this specific aim to determine the effect of the NSC therapy alone. Note that listed above are the times for bolus dosing. However, if the optimal dose is from the alternate days protocol, rats will be administered the treatments on days 7, 9, 11, 13, 15, and 17 for the acute time point, days 21, 23, 25, 27, 29, and 31 for the delayed time, and days 90, 92, 94, 96, 98, and 100 for the chronic period.

Discussion. Motor assessment will include the rotarod task because it, unlike the beam-walk task, is sensitive enough to detect long-term deficits and subsequent recovery as shown after various types of brain trauma. Using the standard beam-walk task would not allow motor testing beyond the one-week time point (acute phase) as the deficits detected by this task will have recovered. Cognitive function will include spatial learning in a well-established water maze task and attentional set-shifting (AST). AST is a sensitive and robust test of executive function. The two tests are proposed so that the potential efficacy of the treatments on various cognitive domains (reference and executive) can be ascertained. The NCS groups will be compared to VEH controls. A successful outcome of these studies would be if any of the behavioral outcomes is significantly better than the non-treated VEH controls. Optimally, these studies will show that executive function can be recovered as it is a long-lasting and devastating disability for TBI patients.

### Example 4

### Determining the Effect of Combining Environmental Enrichment (EE) with NSC Therapy on Motor and Cognitive Benefits (Prophetic)

The NSC regimen used in Example 3 will be combined with a clinically-relevant rehabilitation paradigm of 4 h of EE per day, which has been optimized to mimic patient time in the clinic. The EE receiving groups will be compared to standard-housed (nonenriched) groups in Example 3. The combination will likely be more effective at improving motor and cognitive function than NSC therapy alone.

Protocol. Adult male and normal cycling female rats will undergo a TBI of moderate severity or sham injury and then intranasally administered NSCs or a comparable volume of vehicle as described for Example 3. The rats will be placed in standard (STD) housing prior to treatments and then those randomized to receive EE will be placed in the EE cages for 4-hr per day and then returned to STD cages. Behavioral assessments will be conducted as described in the Methods and then the rats will be sacrificed to determine cell migration. Computational analytical methods applied to optically-cleared brain sections (CLARITY) will be utilized to quantify and validate NSC migration and distribution in TBI vs. sham brains (see Methods Section).

TABLE 3
Determine the effect of combining environmental enrichment
(EE) with NSC therapy on motor and cognitive benefits

| Groups | TBI (M) | TBI (F) | Sham (M) | Sham (F) |
|---|---|---|---|---|
| Optimal NCS dose from | n = 10, | n = 10, | n = 10, | n = 10, |
| Ex. 2 and VEH, day 7 | 10 | 10 | 10 | 10 |
| post-surgery + EE | | | | |
| Optimal NCS dose from | n = 10, | n = 10, | n = 10, | n = 10, |
| Ex. 2 and VEH, day 21 | 10 | 10 | 10 | 10 |
| post-surgery + EE | | | | |
| Optimal NCS dose from | n = 10, | n = 10, | n = 10, | n = 10, |
| Ex. 2 and VEH, day 90 | 10 | 10 | 10 | 10 |
| post-surgery + EE | | | | |

N = 240 * As in Ex. 3, group sizes were determined based on our numerous published behavioral studies showing that 10 rats per group are required to provide sufficient power for behavioral assessments. Note that listed above are the times for bolus dosing. However, if the optimal dose from Ex. 2 is the once every alternate day protocol, rats will be administered the treatments on days 7, 9, 11, 13, 15, and 17 for the acute time point, days 21, 23, 25, 27, 29, and 31 for the delayed time, and days 90, 92, 94, 96, 98, and 100 for the chronic period. EE will begin on the first day of intranasal NSC or VEH administration and continue until all behavioral assessments have been completed. The rats in Ex. 3 will be compared to those in this example to determine the extent that EE further enhanced NSC-induced benefits. As such, this table only lists rats undergoing EE.

Discussion. Numerous studies have shown that the typical EE paradigm of providing EE immediately after TBI and continuing it 24 hr per day through the duration of behavioral testing leads to significant benefits in motor and cognition. However, this approach is not clinically relevant in terms of timing and does not afford the opportunity to evaluate possible additive or synergistic effects of combined treatments because of its robust benefits (Bondi et al., 2014a, 2015). However, it has also been shown that 4-hr of EE per day does not enhance recovery when administered alone but when paired with a therapy that produces benefits the result is greater improvement (de la Tremblaye et al., 2017b). Hence, using the 4-hr of EE per day paradigm to mimic time in the clinic and is thus clinically-relevant (patients often receive ≤4-hr of rehabilitation daily). The EE groups will be compared to the standard-housed (non-enriched) groups from Example 3 to save on adding more standard (STD)-housed rats in this Example. Motor and cognitive function will be assessed as in Example 3. While it is not expected that 4-hr of EE to produce benefits alone, the combination of EE and NSCs will likely synergize to be more effective at improving motor and cognitive function than NSC therapy alone.

### Example 5

### Materials and Methods

Unless specified otherwise, the methods for conducting the studies described in Examples 2-5 are as follows.

LM-NSC008 Cell Culture.: LM-NSC008 cells, stably expressing the L-MYC gene, were generated and characterized (Li et al., 2016). Briefly, LM-NSC008 cells will be cultured in serum-free NSC medium (RHB-A medium; Cell Science) supplemented with 10 ng/mL basic FGF, 10 ng/mL EGF, 2 mM L-glutamine (Invitrogen), Gem21 NeuroPlex Serum-Free Supplement (GeminiBio-Products, #400 ±160), and penicillin-streptomycin (Mediatech, 30-002-CI) as described. LM-NSC008 cells will be plated on 24-well plates at a density of 2×104 cells/cm2 (40,000 cells/well) for conventional cell culture expansion. Similar self-renewal and growth kinetics of LM-NSC008 cells at p5 and P45 have been demonstrated in vitro using IncuCyte (Rockne et al., 2018). Scale-up expansion of LM-NSC008 cells to large cell banks if necessary (expansion of LM-NSC008 cells from 5×107 cells to 3×109 cells within 10 days) will be done using a Quantum Cell Expansion System according to the SOPs and method previously developed (Rockne et al., 2018).

Surgery

Controlled cortical impact (CCI) injury. CCI will be produced as previously described (Dixon et al., 1991; Cheng et al., 2007; Bondi et al., 2014b). Briefly, following anesthesia the rats are secured in a stereotaxic frame, ventilated mechanically, and temperature maintained at 37±0.5° C. Utilizing aseptic procedures a craniectomy is made in the right hemisphere and an impact of 2.8 mm tissue deformation (moderate severity) is provided. Sham rats are not subjected to the impact but receive all other surgical manipulations to control for anesthesia and craniectomy. Estrous stage will be evaluated in females, by vaginal lavage (as per methods in Monaco et al., 2013; Free et al., 2017) at injury to be used as a covariate with outcome.

Post-Surgery

Drug administration. Cyclosporin A (CsA; 10 mg/kg; s.c.) will be provided to all rats two days prior to the first nasal administration of NSC or VEH and once daily until the behavioral regimen is completed. CsA treatment is provided for greater engraftment of human LM-NSC008 cells. To control for the possible protective effects of CsA, all rats will receive the treatment, therefore negating any possible advantages.

Environmental manipulation. Rats designated for EE will be placed in 36×30×20 inch stainless steel-wire cages containing various toys and nesting materials. To maintain novelty, the objects are rearranged every day. Ten rats, which include TBI and sham controls, are housed together. Rats will receive ≤4-hr per day to mimic clinical rehabilitation. Additionally, rats will be exposed to EE beginning on day 7, day 21, or day 90 after TBI or sham surgery to correspond with NSC administration as indicated in Aim 3. The delayed rehabilitation also mimics the clinic. Rats in the standard conditions are placed in standard steel-wire mesh cages (2 rats per cage) with ad libitum food and water. To “blind” the experimenter evaluating behavior from knowing who is receiving EE, another will take the rats to the behavioral room(s).

Motor performance (rotarod). To assess fine motor and long-term function a sensitive rotarod protocol will be used (Hamm et al., 1994; Hamm et al., 1994; Hamm, 2001; Monaco et al., 2013). Rats will be pretrained prior to surgery. On the day of surgery, the rats receive 3 trials to establish a baseline. Testing will be conducted beginning one day after the last NSC or VEH injection and consists of alternate daily trails where duration (maximum 90 sec) and speed (15-80 rpm), before losing balance and falling off are recorded for 21 days as this is when the sham rats are stable to baseline and can be assessed where the treatment groups are at relative to the shams. The average daily scores for each subject are used in the statistical analyses.

Cognitive function. Spatial learning is assessed in a Morris water maze task that is sensitive to cognition after TBI (Hamm et al, 1992; Scheff et al., 1997; Kline et al., 2002a). The pool is situated in a room with salient visual cues. Training consists of 4 daily trials for 5 days to locate the platform and will commence one day after the last NSC or VEH injection (for data for the acute time, this was on day 18, but for the delayed and chronic times [Examples 3 and 4], testing will begin on days 32 and 101). Deficits in the water maze persist up to one year if untreated and thus there is no concern that spontaneous recovery will preclude the efficacy of NSC alone or with EE at the late times. For each block of trials, the rats are randomly placed in the pool from each of the four start locations. Each trial lasts until the rat climbs onto the platform or until 120 s has elapsed. Rats that fail to locate the platform within the allotted time are manually guided to it and allowed to remain on for 30 s. The times of the 4 daily trials for each rat are averaged and used in the statistical analyses. One day after the final acquisition training session all rats are given a single probe trial to measure memory. Estrous stage will be determined in females on the first day of testing as a covariate with functional outcome.

Attentional set-shifting test (AST). To assess executive function and behavioral flexibility via a test that allows rats to engage in natural environment-like rule learning, they will be trained and tested on the AST at the completion of the spatial learning (reference memory) tasks. The AST has been utilized extensively in the field, and was first published after TBI (Bondi et al., 2014b). Briefly, the apparatus is a rectangular Plexiglas arena (30×51×25 cm). The stimulus bowls are small terracotta pots defined by cues along two dimensions (medium in pots and an aromatic odor on the inner rim). The bait, a quarter Cheerio is buried below the surface of the medium in the “positive” pot. Presentation of stimuli will be varied according to the contingency schedule below. The dependent measures are trials to reach criterion, total response errors and set loss errors (i.e., failure to maintain acquisition of correct stimulus contingency after 3 or more correct responses) for each stage. The AST is akin to the Wisconsin Card Sorting task for assessing strategy-switching impairments (executive dysfunction) in humans (Birrell and Brown, 2000).

Brain tissue processing. At euthanasia, all rats will be anesthetized with isoflurane and perfused transcardially with ice cold PBS, pH 7.4. Half of the rats (3 males and 3 females) will be perfused with a Hydrogel Solution; brains will be submerged in 10-20 mL of Hydrogel Solution for 24hr at 4° C. and then will be analyzed by CLARITY and quantification of LM-NSC008s. The remaining brains (male/female) will be fresh frozen, sectioned, stained with DAPI to visualize NSCs by fluorescence microscopy and 3D reconstructed using Reconstruct software (LM-NSC008 cells will be quantified). H&E and immunohistochemical staining (IHC) for human specific neural (PAX6, Tuj1, Neun, MAP 2), glial (GFAP and Stem 123) and oligodendrocyte (O4) markers will be used to visualize LM-NSC008 cells and to determine the fate of these cells in TBI and sham rat brains. IHC will also be performed for immune T cells infiltration into TBI sites (CD4 and CD8) cells to demonstrate dynamic changes of immune cells after TBI in rats (Bai et al., 2017). IHC will be performed on 2D histological sections, with axial or coronal 10-micron sections being prepared after brain harvesting. Sections will be stained with a primary antibodies and detection will be performed using a biotinylated anti-mouse IgG secondary antibody and avidin-biotin complex (ABC) technique, followed by colorimetric detection using DAB. The sections will be counterstained with hematoxylin.

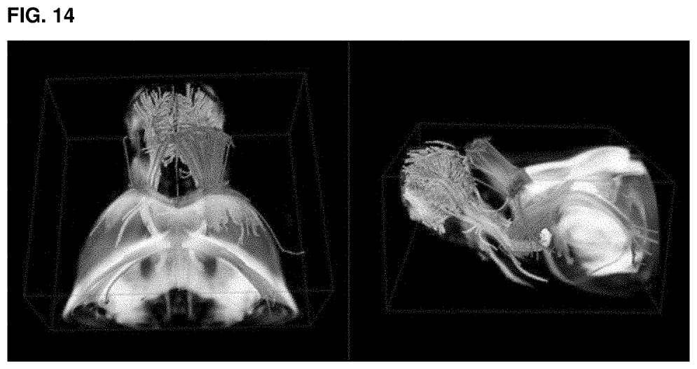

Computational analysis. LM-NSC008 distribution and migration analysis. Volumetric 3D confocal microscopy imaging of CLARITY cleared brains will be analyzed for distribution of fluorescent-labeled LM-NSC008 cells. LM-NSC008s will be identified by e.GFP and white matter tracts by Dil fluorescence intensity. LM-NSC008 distribution will be evaluated relative to WM tracts and anatomical structures as previously described (Budde and Annese, 2013). Tissue orientation will be quantified and used in computational models of NSC migration that will be compared with observed distributions of NSCs as previously described (Rockne et al., 2017). Briefly, the computational prediction model approach is a 3D spatial Markov-Chain-Monte-Carlo probabilistic model of migration in which the edges and nodes of the migration path are determined by the orientation and anisotropy of the tissue. This predicts a preferential migration along WM tracts and alignment with brain tissue structures. The probabilistic nature of this algorithm produces distributions of paths. The paths within one standard deviation from the mean across all paths from an initial site will be used as the most likely path. Distribution of NSCs will be evaluated within the predicted path and margin within the brain. The feasibility of this approach in LM-NSC008 endogenous migration in the rodent brain has already been demonstrated (see, e.g., FIG. 14).

White matter integrity analysis. Luxol Fast Blue and markers of axonal injury (APP, amino cupric silver) will be used to identify myelinated structures in the brain. Image processing algorithms of 3D confocal microscopy of CLARITY-cleared brains will be used to assess WM integrity along with pathological analysis.

Behavioral analyses are performed on data collected by observers blinded to conditions using Statview 5.0.1 software. The behavioral data will be analyzed by repeated measures ANOVA or by one-factor ANOVAs as appropriate. When the overall ANOVA reveals a significant effect, the data will be further analyzed with the Bonferroni/Dunn post-hoc test to determine specific group differences as it corrects for multiple comparisons. A mixed effects multivariate regression modeling approach will be used when appropriate. Power analyses for behavior assume 80% power, type 1 error rate of 0.05 and two-sided alternative hypothesis. For behavioral studies with 10 rats per group, an ANOVA will be able to detect an effect size of 0.33 between groups, 0.39 for time, and 0.35 for the interaction effect of group and time. Statistical analysis for NSCs will be performed by Liu Xueli, PhD from the core facility at City of Hope (support letter attached). Statistical analyses will be performed separately for each NSC dose and schedule of administration using SAS version 9.4 and Prism version 6. Results will be analyzed using random intercept and slope regression models with intensities analyzed on a log scale. Models will include quadratic time, group, and their interactions. Significant estimated group differences from curves will be analyzed using t-tests. In addition, t-tests on raw data will be used to confirm earliest time point of difference among groups. For biodistribution studies groups 12 rats per group (6 male and 6 female) will provide at least 80% power to distinguish an increase of 0.50 in the event rate of testing group (0.55) over any control group (0.05) individually in quantification of LM-NSC008s using one-tailed Fischer's exact test at 0.05 level of significance. To be conservative and protective, multiple testing problems will be ignored and P-value will not be adjusted.

From the foregoing, it will be appreciated that specific embodiments of the invention have been described herein for purposes of illustration, but that various modifications may be made without deviating from the scope of the invention. Accordingly, the invention is not limited except as by the appended claims.

### REFERENCES

All references cited below and in the disclosure are hereby incorporated by reference in their entirety, as if fully set forth herein.

Aboody K S, Najbauer J, Metz M Z, D'Apuzzo M, Gutova M, Annala A J, Synold T W, et al. Neural stem cell-mediated enzyme/prodrug therapy for glioma: preclinical studies.Sci Transl Med 5(184):184ra159.

Bai R, Gao H, Han Z. Huang S, Ge X, Chen F, Lei P. Flow cytometric characterization of T cell subsets and microglia after repetitive mild traumatic brain injury in rats. Neurochem Res 42:2892-2901, 2017.

Balyasnikova I V, Prasol M S, Ferguson S D, Han Y, Ahmed A U, Gutova M, Tobias A L, et al. Intranasal delivery of mesenchymal stem cells significantly extends survival of irradiated mice with experimental brain tumors. Mol Ther 22:140-148, 2014.

Barish M E, Herrmann K, Tang Y, Argalian Herculian S, Metz M, Aramburo S, Tirughana R, Gutova M, et al. Human neural stem cell Biodistribution and predicted tumor coverage by a diffusible therapeutic in a mouse glioma model.Stem Cells Transl Med 6:1522-1532, 2017.

Binder L. Persistent symptoms after mild head injury: a review of the postconcussive syndrome. J Clin Exp Neuropsychol 8:323-346, 1986.

Birrell J M, Brown V J. Medial frontal cortex mediates perceptual attentional set shifting in the rat. J Neurosci. 20:4320-4324, 2000.

Blackerby W F. Intensity of rehabilitation and length of stay. Brain Inj 4:167-173, 1990.

Bleimeister, I. H., et al., Environmental enrichment and amantadine confer individual but nonadditive enhancements in motor and spatial learning after controlled cortical impact injury. Brain Res, 2019. 1714: p. 227-233.

Bogdanova, Y., et al., Computerized Cognitive Rehabilitation of Attention and Executive Function in Acquired Brain Injury: A Systematic Review. J Head Trauma Rehabil, 2016. 31(6): p. 419-433.

Bondi C O, Klitsch K C, Leary J B, Kline A E. Environmental enrichment as a viable neurorehabilitation strategy for experimental traumatic brain injury. J Neurotrauma, 31:873-888, 2014a.

Bondi C O, Cheng J P, Tennant H M, Monaco C M, Kline A E. Old dog, new tricks: the attentional set-shifting test as a novel cognitive behavioral task after controlled cortical impact injury. J Neurotrauma, 31:926-937, 2014b.

Bondi, C. O., et al., Adolescent behavior and dopamine availability are uniquely sensitive to dietary omega-3 fatty acid deficiency. Biol Psychiatry, 2014c. 75(1): p. 38-46.

Bondi C O, Semple B D, Noble-Haeusslein L J, Osier N D, Carlson S W, Dixon C E, Giza C C, Kline A E. Found in translation: understanding the biology and behavior of traumatic brain injury. Neurosci Biobehav Revs., 58:123-146, 2015.

Brain Res Mol Brain Res 53:285-290, 1998. Rockne RC, Tsaturyan L, Adhikarla V, Li Z, Aboody K S, Barish M E, Gutova M. Development of a computational prediction model for migration of exogenous L-myc expressing neural stem cell in the brain. ISSCR International Symposium. Basel, Switzerland (2017).

Budde M D, and Annese J. Quantification of anisotropy and fiber orientation in human brain histological sections. Front Integr Neurosci 7: 3, 2013.

Chen X, Li Y, Kline A E, Dixon C E, Zafonte R D, Wagner A K. Gender and environmental effects on regional brain-derived neurotrophic factor expression after experimental traumatic brain injury. Neuroscience 135(1):11-17, 2005.

Cheng J P, Aslam H A, Hoffman A N, Zafonte R D, Kline A E. The neurobehavioral benefit conferred by a single systemic administration of 8-OH-DPAT after brain trauma is confined to a narrow therapeutic window. Neurosci Lett 416:165-168, 2007.

Cheng J P, Hoffman A N, Zafonte R D, Kline A E. A delayed and chronic treatment regimen with the 5-HT1A receptor agonist 8-OH-DPAT after cortical impact injury facilitates motor recovery and acquisition of spatial learning. Behav Brain Res 194:79-85, 2008.

Chirumamilla, S., et al., Traumatic brain injury induced cell proliferation in the adult mammalian central nervous system. J Neurotrauma, 2002. 19(6): p. 693-703.

Cox, C. S., Jr., Cellular therapy for traumatic neurological injury. Pediatr Res, 2018. 83(1-2): p.325-332.

de la Tremblaye P B, Bondi C O, Lajud N, Cheng J P, Radabaugh H L, Kline A E. Galantamine and environmental enrichment enhance cognitive recovery after experimental traumatic brain injury, but do not confer additional benefits when combined. J Neurotrauma, 34:1610-1622, 2017a.

de la Tremblaye P B, Cheng J P, Bondi C O, Kline A E. Environmental enrichment, alone or in combination with various pharmacotherapies, confers marked benefits after traumatic brain injury. Neuropharmacology 145:13-24, 2019.

de la Tremblaye P B, Wellcome J L, de Witt B W, Cheng J P, Skidmore E R, Bondi C O, Kline A E. Rehabilitative success after brain trauma by augmenting a sub-therapeutic dose of environmental enrichment with galantamine. Neurorehabil Neural Repair, 31:977-985, 2017b.

de Witt B W, Ehrenberg K M, McAloon R L, Panos A H, Shaw K E, Raghavan P V, Skidmore E R, Kline A E. Abbreviated environmental enrichment enhances neurobehavioral recovery comparably to continuous exposure after traumatic brain injury. Neurorehabil Neural Repair 25:343-350, 2011.

Dixon C E, Clifton G L, Lighthall J W, Yaghmai A A, Hayes R L. A controlled cortical impact model of traumatic brain injury in the rat. J Neurosci Methods 39:253-262, 1991.

Dixon C E, Kochanek P M, Yan H Q, Schiding J K, Griffith R G, Baum E, Marion D W, DeKosky S T. One-year study of spatial memory performance, brain morphology and cholinergic markers after moderate controlled cortical impact in rats. J Neurotrauma 16:109-122, 1999.

Doetsch, F., J. M. Garcia-Verdugo, and A. Alvarez-Buylla, Cellular composition and three-dimensional organization of the subventricular germinal zone in the adult mammalian brain. J Neurosci, 1997. 17(13): p. 5046-61.

Doppenberg E M R, Choi S C, Bullock R. Clinical trials in traumatic brain injury: lessons for the future. J Neurosurg Anesthesiol 16:87-94, 2004.

Duncan, K., et al., Stem cell-paved biobridges facilitate stem transplant and host brain cell interactions for stroke therapy. Brain Res, 2015. 1623: p. 160-5.

Faul M, Xu L, Wald M M, Coronado V G. Traumatic brain injury in the United States: emergency department visits, hospitalizations and deaths 2002-2006. Atlanta (GA): Centers for Disease Control and Prevention, National Center for Injury Prevention and Control, 2010.

Feng Z, Gao F. Stem cell challenges in the treatment of neurodegenerative disease. CNS Neurosci Ther 18:142-148, 2012.

Free K E, Greene A M, Bondi C O, Lajud N, de la Tremblaye P B, Kline A E. Comparable impediment of cognitive function in female and male rats subsequent to daily administration of haloperidol after traumatic brain injury. Exp Neurol., 296:62-68, 2017.

Frick K M, Fernandez S M. Enrichment enhances spatial memory and increases synaptophysin levels in aged female mice. Neurobiol Aging 24:615-626, 2003.

Gage, F. H. and S. Temple, Neural stem cells: generating and regenerating the brain. Neuron, 2013. 80(3): p. 588-601.

Gage, F. H., Brain, repair yourself. Sci Am, 2003. 289(3): p. 46-53.

Gage, F. H., J. Ray, and L. J. Fisher, Isolation, characterization, and use of stem cells from the CNS. Annu Rev Neurosci, 1995. 18: p. 159-92.

Gage, F. H., Neuronal stem cells: their characterization and utilization. Neurobiol Aging, 1994. 15 Suppl 2: p. S191.

Garcia A N, Shah M A, Dixon C E, Wagner A K, Kline A E. Biologic and plastic effects of experimental traumatic brain injury treatment paradigms and their relevance to clinical rehabilitation. PMR 3:S18-S27, 2011.

Gennai, S., et al., Cell-based therapy for traumatic brain injury. Br J Anaesth, 2015. 115(2): p.203-12.

Gentile A M, Beheshti Z, Held J M. Enrichment versus exercise effects on motor impairments following cortical removals in rats. Behav Neural Biol 47:321-332, 1987.

Giza C C, Griesbach G S, Hovda D A. Experience-dependent behavioral plasticity is disturbed following traumatic injury to the immature brain. Behav Brain Res 157:11-22, 2005.

Goldstein M. Traumatic brain injury: a silent epidemic. Ann Neurol 27:327, 1990.

Gutova M, Goldstein L, Metz M, Hovsepyan A, Tsurkan L G, Tirughana R, Tsaturyan L, et al. Optimization of a neural stem-cell-mediated carboxylesterase/Irinotecan gene therapy for metastatic neuroblastoma. Mol Ther Oncolytics 4:67-76, 2017.

Gutova M, Najbauer J, Frank R T, Kendall S E, Gevorgyan A, Metz M Z, Guevorkian M, et al. Urokinase plasminogen activator and urokinase plasminogen activator receptor mediate human stem cell tropism to malignant solid tumors. Stem Cells 26:1406-1413, 2008.

Gutova M, Shackleford G M, Khankaldyyan V, Herrmann K A, Shi X H, Mittelholtz K, Abramyants Y, et al. Neural stem cell-mediated CE/CPT-11 enzyme/prodrug therapy in transgenic mouse model of intracerebellar medulloblastoma. Gene Ther 20:143-150, 2013a.

Gutova, M., et al., Magnetic Resonance Imaging Tracking of Ferumoxytol-Labeled Human Neural Stem Cells: Studies Leading to Clinical Use. Stem Cells Transl Med, 2013b. 2(10): p. 766-775.

Gutova M, Shahmanyan D, Oganesyan D, Abramyants Y, Danielyan L, Frey W H II, Khankaldyyan, et al. Intranasal delivery of therapeutic neural stem cells to target intracerebral glioma. J Stem Cells Regen Med 1:007, 2015.

Gutova, M., et al., Intranasal Delivery of Therapeutic Neural Stem Cells to Target Intracerebral Glioma. J Stem Cells Regen Med, 2015. 1: p. 007.

Hamm R J, Dixon C E, Gbadebo D M, Singha A K, Jenkins L W, Lyeth B G, Hayes R L. Cognitive deficits following traumatic brain injury produced by controlled cortical impact. J Neurotrauma 9:11-20, 1992.

Hamm R J, Pike B R, O'Dell D M, Lyeth B G, Jenkins L W. The rotarod test: an evaluation of its effectiveness in assessing motor deficits following traumatic brain injury. J Neurotrauma 11:187-196, 1994.

Hamm R J, Temple M D, O'Dell D M, Pike B R, Lyeth B G. Exposure to environmental complexity promotes recovery of cognitive function after traumatic brain injury. J Neurotrauma 13:41-47, 1996.

Hamm R J. Neurobehavioral assessment of outcome following traumatic brain injury in rats: an evaluation of selected measures. J Neurotrauma 18:1207-1216, 2001.

Haus, D. L., et al., Transplantation of human neural stem cells restores cognition in an immunodeficient rodent model of traumatic brain injury. Exp Neurol, 2016. 281: p. 1-16.

Held J M, Gordon J, Gentile A M. Environmental influences on locomotor recovery following cortical lesions in rats. Behav Neurosci 99:678-690, 1985.

Hicks R R, Zhang L, Atkinson A, Stevenon M, Veneracion M, Seroogy, K B. Environmental enrichment attenuates cognitive deficits, but does not alter neurotrophin gene expression in the hippocampus following lateral fluid percussion brain injury. Neuroscience 3:631-637, 2002.

Hoffman A N, Malena R R, Westergom B P, Luthra P, Cheng J P, Aslam H A, Zafonte R D, Kline A E. Environmental enrichment-mediated functional improvement after experimental traumatic brain injury is contingent on task-specific neurobehavioral experience. Neurosci Lett 431:226-230, 2008.

Horneman G, Emanuelson I. Cognitive outcome in children and young adults who sustained a severe and moderate traumatic brain injury 10 years earlier. Brain Inj 23:907-914, 2009.

Hyder A A, Wunderlich C A, Puvanachandra P, Gururaj G, Kobusingye O C. The impact of traumatic brain injuries: a global perspective. NeuroRehabilitation 22:341-353, 2007.

Kline A E, Massucci J L, Dixon C E, Zafonte R D, Bolinger B D. The therapeutic efficacy conferred by the 5 HT1A receptor agonist 8-hydroxy-2-(di-n-propylamino)tetralin (8-OH-DPAT) after experimental traumatic brain injury is not mediated by concomitant hypothermia. J Neurotrauma 21:175-185, 2004a.

Kline A E, Massucci J L, Ma X, Zafonte R D, Dixon C E. Bromocriptine reduces lipid peroxidation and enhances spatial learning and hippocampal neuron survival in a rodent model of focal brain trauma. J Neurotrauma 21:1712-1722, 2004b.

Kline A E, Massucci J L, Marion D W, Dixon C E. Attenuation of working memory and spatial acquisition deficits after a delayed and chronic bromocriptine treatment regimen in rats subjected to traumatic brain injury by controlled cortical impact. J Neurotrauma 19:415-425, 2002a.

Kline A E, McAloon R L, Henderson K A, Bansal U K, Ganti B M, Ahmed R H, Gibbs R B, Sozda C N. Evaluation of a combined therapeutic regimen of 8-OH-DPAT and environmental enrichment after experimental traumatic brain injury. J Neurotrauma 27:2021-2032, 2010.

Kline A E, Wagner A K, Westergom B P, Malena R R, Zafonte R D, Olsen A S, Sozda C N, Luthra P, Panda M, Cheng J P, Aslam H A. Acute treatment with the 5-HT1A receptor agonist 8-OH-DPAT and chronic environmental enrichment confer neurobehavioral benefit after experimental brain trauma. Behav Brain Res 177:186-194, 2007.

Kline A E, Yu J, Horvath E, Marion D W, Dixon C E. The selective 5-HT1A receptor agonist repinotan HCI attenuates histopathology and spatial learning deficits following traumatic brain injury in rats. Neuroscience 106:547-555, 2001.

Kline A E, Yu J, Massucci J L, Zafonte R D, Dixon C E. Protective effects of the 5-HT1A receptor agonist 8-hydroxy-2-(di-n-propylamino)tetralin (8-OH-DPAT) against traumatic brain injury-induced cognitive deficits and neuropathology in adult male rats. Neurosci Lett 333:179-82, 2002b.

Kline, A. E., et al., Acute systemic administration of interleukin-10 suppresses the beneficial effects of moderate hypothermia following traumatic brain injury in rats. Brain Res, 2002c. 937(1-2): p. 22-31.

Kline, A. E., et al., Combination therapies for neurobehavioral and cognitive recovery after experimental traumatic brain injury: Is more better? Prog Neurobiol, 2016. 142: p. 45-67.

Kokiko O N, Hamm R J. A review of pharmacological treatments used in experimental models of traumatic brain injury. Brain Inj 21:259-274, 2007.

Kutten K S, V. J., Charon N, Ye L, Deisseroth K, Miller MI. (2016). Deformably Registering and Annotating Whole CLARITY Brains to an Atlas via Masked LDDMM. ArXiv.

Lajud, N., et al., Delayed and Abbreviated Environmental Enrichment after Brain Trauma Promotes Motor and Cognitive Recovery That Is Not Contingent on Increased Neurogenesis. J Neurotrauma, 2019. 36(5): p. 756-767.

Leary J B, Bondi C O, LaPorte M J, Carlson L J, Radabaugh H L, Cheng J P, Kline A E. The therapeutic efficacy of environmental enrichment and methylphenidate alone and in combination after controlled cortical impact injury. J Neurotrauma, 34:444-450, 2017.

Li Z, Oganesyan D, Mooney R, Rong X, Christensen M J, Shahmanyan D, Perrigue P M, et al. L-MYC expression maintains self-renewal and prolongs multipotency of primary human neural stem cells. Stem Cell Reports 7:483-495, 2016.

Loebinger M R, Kyrtatos P G, Turmaine M, Price A N, Pankhurst Q, Lythgoe M F, Janes S M. Magnetic resonance imaging of mesenchymal stem cells homing to pulmonary metastases using biocompatible magnetic nanoparticles. Cancer Res 69:8862-8867, 2009.

Lois, C. and A. Alvarez-Buylla, Proliferating subventricular zone cells in the adult mammalian forebrain can differentiate into neurons and glia. Proc Natl Acad Sci U S A, 1993. 90(5): p. 2074-7.

Mahmood A, Goussev A, Lu D, Qu C, Xiong Y, Kazmi H, Chopp M. Long-lasting benefits after treatment of traumatic brain injury (TBI) in rats with combination therapy of marrow stromal cells (MSCs) and simvastatin. J Neurotrauma 25:1441-1447, 2008.

Marchetto M C, Carromeu C, Acab A, Yu D, Yeo G W, Mu Y, Chen G. A model for neural development and treatment of Rett syndrome using human induced pluripotent stem cells. Cell 143:527-539, 2010.

Marchetto M C, Winner B, Gage F H. Pluripotent stem cells in neurodegenerative and neurodevelopmental diseases. Hum Mol Genet 19(R1):R71-76, 2010.

Max W, Mackenzie E J, Rice D P. Head injuries: costs and consequences. J Head Trauma Rehabil 6:76-91, 1991.S

Menon D K. Unique challenges in clinical trials in traumatic brain injury. Crit Care Med 37:S129-S135, 2009.

Millis S R, Rosenthal M, Novack T A, Sherer M, Nick T G, Kreutzer J S, High Jr. W M, Ricker J K. Long-term neuropsychological outcome after traumatic brain injury. J Head Trauma Rehabil 16:343-355, 2001.

Monaco C M, Mattiola V V, Folweiler K A, Tay J K, Yelleswarapu N K, Curatolo L M, Matter A M, Cheng J P, Kline A E. Environmental enrichment promotes robust functional and histological benefits in female rats after controlled cortical impact injury. Exp Neurol., 247:410-418, 2013.

Najbauer, J., et al., Cellular host responses to gliomas. PLoS One, 2012. 7(4): p. e35150.

Nakagawa M, Takizawa N, Narita M, Ichisaka T, Yamanaka S. Promotion of direct reprogramming by transformation-deficient Myc. Proc Natl Acad Sci U S A 107:14152-14157, 2010.

Nudi E T, Jacqmain J, Dubbs K, Geeck K, Salois G, Searles M A, Smith J S. Combining enriched environment, progesterone, and embryonic neural stem cell therapy improves recovery following brain injury. J Neurotrauma 32:1117-1129, 2015.

Parton A, Coulthard E, Husain M. Neuropharmacological modulation of cognitive deficits after brain damage. Curr Opin Neurol 18:675-680, 2005.

Passineau M J, Green E J, Dietrich W D. Therapeutic effects of environmental enrichment on cognitive function and tissue integrity following severe traumatic brain injury in rats. Exp Neurol 168:373-384, 2001.

Peruzzaro S T, Gallagher J, Dunkerson J, Fluharty S, Mudd D, Hoane M R, Smith J S. The impact of enriched environment and transplantation of murine cortical embryonic stem cells on recovery from controlled cortical contusion injury. Restor. Neurol. Neurosci. 31:431-450, 2013.

Radabaugh H L, Carlson L J, O′Neil D A, LaPorte M J, Monaco C M, Cheng J P, de la Tremblaye P B, Lajud N, Bondi C O, Kline A E. Abbreviated environmental enrichment confers neurobehavioral, cognitive, and histological benefits in brain-injured female rats. Exp Neurol., 286:61-68, 2016.

Rasmuson S, Olsson T, Henriksson B G, Kelly P A, Holmes M C, Seckl J R, Mohammed A H. Environmental enrichment selectively increases 5-HT1A receptor mRNA expression and binding in the rat hippocampus.

Rice, H. E., et al., Superparamagnetic iron oxide labeling and transplantation of adipose-derived stem cells in middle cerebral artery occlusion-injured mice. AJR Am J Roentgenol, 2007. 188(4): p. 1101-8.

Rockne R C, Adhikarla V, Tsaturyan L, Li Z, Masihi M B, Aboody K S, Barish M E, Gutova M. Long-term stability and computational analysis of migration patterns of L-MYC immortalized neural stem cells in the brain. PLoS One 13: e0199967, 2018.

Rolfe, A. and D. Sun, Stem Cell Therapy in Brain Trauma: Implications for Repair and Regeneration of Injured Brain in Experimental TBI Models, in Brain Neurotrauma: Molecular, Neuropsychological, and Rehabilitation Aspects, F.H. Kobeissy, Editor. 2015: Boca Raton (Fla.).

Rose F D, Davey M J, Love S, Dell P A. Environmental enrichment and recovery from contralateral sensory neglect in rats with large unilateral neocortical lesions. Behav Brain Res 24:195-202, 1987.

Scheff S W, Baldwin S A, Brown R W, Kraemer P J. Morris water maze deficits in rats following traumatic brain injury: lateral controlled cortical impact. J Neurotrauma 14:615-627, 1997.

Selassie A W, Zaloshnja E, Langlois J A, Miller T, Jones P, Steiner C. Incidence of long-term disability following traumatic brain injury hospitalization, United States, 2003. J Head Trauma Rehabil 23:123-131, 2008.

Shiel A, Burn J P, Henry D, Clark J, Wilson B A, Burnett M E, McLellan D L. The effects of increased rehabilitation therapy after brain injury: results of a prospective controlled trial. Clin Rehabil 15:501-514, 2001.

Sosin D M, Sniezek J E, Waxweiler R J. Trends in death associated with traumatic brain injury, 1979 through 1992. JAMA 273:1778-1780, 1995.

Sozda C N, Hoffman A N, Olsen A S, Cheng J P, Zafonte R D, Kline A E. Empirical comparison of typical and atypical environmental enrichment paradigms on functional and histological outcome after experimental traumatic brain injury. J Neurotrauma 27:1047-1057, 2010.

Spurlock M S, Ahmed Al, Rivera K N, Yokobori S, Lee S W, Sam P N, Shear D A, et al. Amelioration of penetrating ballistic-like brain injury induced cognitive deficits after neuronal differentiation of transplanted human neural stem cells. J Neurotrauma 34:1981-1995, 2017.

Summers C R, Ivins B, Schwab K A. Traumatic brain injury in the United States: an epidemiologic overview. Mt Sinai J Med 76:105-110, 2009.

Taylor C A, Bell J M, Breiding M J, Xu L. Traumatic brain injury—related emergency department visits, hospitalizations, and deaths — United States, 2007 and 2013. MMWR Surveill Summ. 66(No. SS-9):1-16, 2017a.

Taylor, D. N., F. J. Ponce, and S. J. Dyess, Survey of Primary Contact Medical and Chiropractic Clinicians on Self-Reported Knowledge and Recognition of Mild Traumatic Brain Injuries: A Pilot Study. J Chiropr Med, 2017b. 16(1): p. 19-30.

Tirughana R, Metz M Z, Li Z, Hall C, Hsu D, Beltzer J, Annala A J, et al. GMP Production and Scale-Up of Adherent Neural Stem Cells with a Quantum Cell Expansion System. Mol Ther Methods Clin Dev 10:48-56, 2018.

Treweek J B, Chan K Y, Flytzanis N C, Yang B, Deverman B E, Greenbaum A, Lignell A, et al., Whole-body tissue stabilization and selective extractions via tissue-hydrogel hybrids for high-resolution intact circuit mapping and phenotyping. Nat Protoc 10:1860-1896, 2015.

van Praag K, Kempermann G, Gage F H. Neural consequences of environmental enrichment. Nat Rev 1:191-198, 2000.

Vanderploeg R D, Schwab K, Walker W C, Fraser J A, Sigford B J, Date E S, Scott S G, Curtiss G, Salazar A M, Warden D L. Rehabilitation of traumatic brain injury in active duty military personnel and veterans: defense and veterans brain injury center randomized controlled trial of two rehabilitation approaches. Arch Phys Med Rehabil 89:2227-2238, 2008.

Wagner A K, Sokoloski J E, Ren D, Chen X, Khan A S, Zafonte R D, Michael A C, Dixon C E. Controlled cortical impact injury affects dopaminergic transmission in the rat striatum. J Neurochem 95:457-465, 2005.

Weston, N M, Sun, D. The potential of stem cells in treatment of traumatic brain injury. Curr Neurol Neurosci Rep 18:1, 2018.

Wheaton P, Mathias J L, Vink R. Impact of early pharmacotherapy treatment on cognitive and behavioral outcome after traumatic brain injury in adults: a meta-analysis. J Clin Psychopharmacol 29:468-477, 2009.

Yang B, Treweek J B, Kulkarni R P, Deverman B E, Chen C K, Lubeck E, Shah S, et al. Single-cell phenotyping within transparent intact tissue through whole-body clearing. Cell 158:945-958, 2014.

Zhang Z, Wang Z, Rui W, Wu S. Magnesium lithospermate B promotes proliferation and differentiation of neural stem cells in vitro and enhances neurogenesis in vivo. Tissue Cell 53:8-14, 2018.

Zhu X L, Poon W S, Chan C C H, Chan S S H. Does intensive rehabilitation improve the functional outcome of patients with traumatic brain injury (TBI)? A randomized controlled trial. Brain Inj 21:681-690, 2007.

Zibara K, Bailout N, Mondello S, Karnib N, Ramadan N, Omais S, Nabbouh A, et al. Combination of drug and stem cells neurotherapy: potential interventions in neurotrauma and traumatic brain injury. Neuropharmacology 145(Pt B): 177-198, 2019.

I/we claim:

1. A method for treating traumatic brain injury (TBI) comprising administering a population of neural stem cells (NSCs) capable of expressing L-MYC to a subject having a TBI, wherein the population of NSCs are administered intranasally.

2. The method of claim 1, further comprising providing environmental enrichment (EE) to the subject in conjunction with or in combination with the NSC administration.

3. The method of claim 1, wherein the population of NSCs are from an LM-NSC008 cell line.

4. The method of claim 2, wherein the EE is provided prior to administering the NSCs, at approximately the same time as the NSC administration, or after the administration of the NSCs.

5. A method for treating traumatic brain injury (TBI) comprising
administering neural stem cells (NSCs) to a subject having a TBI; and
providing environmental enrichment (EE) to the subject in conjunction with or in combination with the NSC administration.

6. The method of claim 5, wherein the population of NSCs express L-MYC.

7. The method of claim 5, wherein the population of NSCs are from an LM-NSC008 cell line.

8. The method of claim 5, wherein the NSCs express are administered intranasally.

9. The method of claim 5, wherein the EE is provided prior to administering the NSCs, at approximately the same time as the NSC administration, or after the administration of the NSCs.
